(19) United States
(12) Patent Application Publication
(10) Pub. No.: US 2009/0092675 A1
(43) Pub. Date: Apr. 9, 2009

(54) COMPOSITIONS CONTAINING MULTIPLE POLYMERS AND PARTICLES MADE USING THE COMPOSITIONS

(75) Inventors: Sonali Puri, Ashland, MA (US); Sharon Mi Tan, Boston, MA (US)

Correspondence Address: FISH & RICHARDSON PC, P.O. BOX 1022, MINNEAPOLIS, MN, 55440-1022, US

(73) Assignee: Boston Scientific Scimed, Inc., Maple Grove, MN (US)

(21) Appl. No.: 12/200,211

(22) Filed: Aug. 28, 2008

Related U.S. Application Data
(60) Provisional application No. 60/977,892, filed on Oct. 5, 2007.

(51) Int. Cl. A61K 9/14; A61P 35/00

(52) U.S. Cl. 424/501

(57) ABSTRACT

The compositions described herein include a first polymer that is either a polyvinyl alcohol or a polyvinyl formal, and a second polymer that is one of a polyvinyl alcohol, a polyvinyl formal, polyvinylpyrrolidone, a polysaccharide, or a polymethacrylate. The first polymer and the second polymer in the composition are different. These compositions are useful in the formation of particles, such as embolic particles, or other medical devices. The compositions are also useful in the delivery of therapeutic agents. Different ratios of the first polymer to the second polymer can provide different rates of release of the therapeutic agent from the composition.

### CROSS-REFERENCE TO RELATED APPLICATION

This application claims priority under 35 U.S.C. §119 to U.S. Ser. No. 60/977,892, filed Oct. 5, 2007, the contents of which are hereby incorporated by reference.

### FIELD

The technology described herein relates to compositions containing multiple polymers and article made using the compositions.

### BACKGROUND

Particles made from polymer compositions can be used to create therapeutic vascular occlusions, which are used to prevent or to treat certain pathological conditions in the body. For example, in therapeutic vascular occlusions (sometimes called “embolizations”), particle compositions can be used to block, or occlude, vessels in the body. As further examples, particle compositions can be used to block microvascular supplies of blood to tumors (thereby depriving the tumors of resources to grow), or to block hemorrhagic conditions in the body (thereby reducing or stopping bleeding).

### SUMMARY

Compositions for making embolic particles, embolic particle chains, and other medical devices, as well as methods for making the same are described herein. The embolic particles, embolic particle chains, and other medical devices can optionally include one or more therapeutic agents.

In one aspect, particles are described herein that include a first polymer that is either a polyvinyl alcohol or a polyvinyl formal, and a second polymer that is either a polyvinyl alcohol, a polyvinyl formal, polyvinylpyrrolidone, a polysaccharide, or a polymethacrylate. The first polymer and the second polymer of this particle are different. The particle can also include a third polymer that is either a polyvinyl alcohol, a polyvinyl formal, polyvinylpyrrolidone, a polysaccharide, or a polymethacrylate. The third polymer, if present, is different from the first and second polymers. Additionally, such a particle can be connected by a link to another particle.

In another aspect, a method for forming a particle is described. In this method, a first polymer is combined with a second polymer to form a polymer composition. The first polymer is either a polyvinyl alcohol or a polyvinyl formal, and a second polymer that is either a polyvinyl alcohol, a polyvinyl formal, polyvinylpyrrolidone, a polysaccharide, or a polymethacrylate. The first polymer and the second polymer of polymer composition are different. Next a particle is formed from the polymer composition. In this method, a therapeutic agent can be added when the first polymer and second polymer are combined, by exposing the polymer composition to the therapeutic agent prior to forming the particle, or by exposing the particle to a therapeutic agent after the polymer composition is formed. The polymer composition can also include a third polymer that is either a polyvinyl alcohol, a polyvinyl formal, polyvinylpyrrolidone, a polysaccharide, or a polymethacrylate. The third polymer, if present, is different from the first and second polymers. Methods of forming the particle from the particle composition include forming the particle in a mold and forming a drop containing the polymer composition and a gelling precursor, and then contacting the drop with a gelling agent.

In a further aspect, a composition for the controlled release of a therapeutic agent is described. This composition includes a first polymer that is either a polyvinyl alcohol or a polyvinyl formal; a second polymer that is either a polyvinyl alcohol, a polyvinyl formal, polyvinylpyrrolidone, a polysaccharide, or a polymethacrylate; and a therapeutic agent. The first polymer and the second polymer of this composition are different. In this composition, different ratios of the first polymer to the second polymer provide different rates of release of the therapeutic agent from the composition. The polymer composition can also include a third polymer that is either a polyvinyl alcohol, a polyvinyl formal, polyvinylpyrrolidone, a polysaccharide, or a polymethacrylate. The third polymer, if present, is different from the first and second polymers.

In another aspect, a method for making a composition is described. In this method, a first polymer is combined with a second polymer. The first polymer is either a polyvinyl alcohol or a polyvinyl formal, and a second polymer that is either a polyvinyl alcohol, a polyvinyl formal, polyvinylpyrrolidone, a polysaccharide, or a polymethacrylate. The first polymer and the second polymer of composition are different. This method provides a composition in which different ratios of the first polymer to the second polymer provide different rates of release of the therapeutic agent from the composition. In this method, a therapeutic agent can be added when the first polymer and second polymer are combined or by exposing the polymer composition to a therapeutic agent after the polymer composition is formed. The polymer composition can also include a third polymer that is either a polyvinyl alcohol, a polyvinyl formal, polyvinylpyrrolidone, a polysaccharide, or a polymethacrylate. The third polymer, if present, is different from the first and second polymers.

### BRIEF DESCRIPTION OF THE FIGURES

FIG. 1A is an illustration of an embodiment of an embolic particle.

FIG. 1B is a cross-sectional view at line 1B of an embodiment of an embolic particle as shown in FIG. 1A.

FIG. 1C is a cross-sectional view of an embodiment of an embolic particle that includes a core.

FIG. 1D is an illustration of an embodiment of a particle chain.

FIG. 2 is a graph showing the release of ketorolac tromethamine from a 100% polyvinyl alcohol film over time.

FIG. 3 is a graph showing the release of ketorolac tromethamine from a 100% polyvinyl formal film over time.

FIG. 4 is an illustration of a cross-sectional view of an embodiment of an embolic particle that is coated with a composition as described herein.

FIG. 5 is an illustration of a cross-sectional view of an embodiment of an embolic particle that includes a coating.

FIGS. 6A-6C are an illustration of an embodiment of a system and method for producing particles.

FIG. 7 is an illustration of an embodiment of a drop generator.

FIGS. 8A and 8B are illustrations of an embodiment of a system and method for producing particles.

FIG. 9 is an illustration of a cross-sectional view of an embodiment of a particle mold.

FIG. 10A is a schematic illustrating an embodiment of injection of an embolic composition into a vessel, and FIG. 10B is an enlarged view of the region 10B in FIG. 10A.

FIG. 11 is graph showing the release of ketorolac tromethamine from various polymer compositions containing polyvinyl alcohol over time.

FIG. 12 is graph showing the release of ketorolac tromethamine from various polymer compositions containing polyvinyl formal over time.

### DETAILED DESCRIPTION

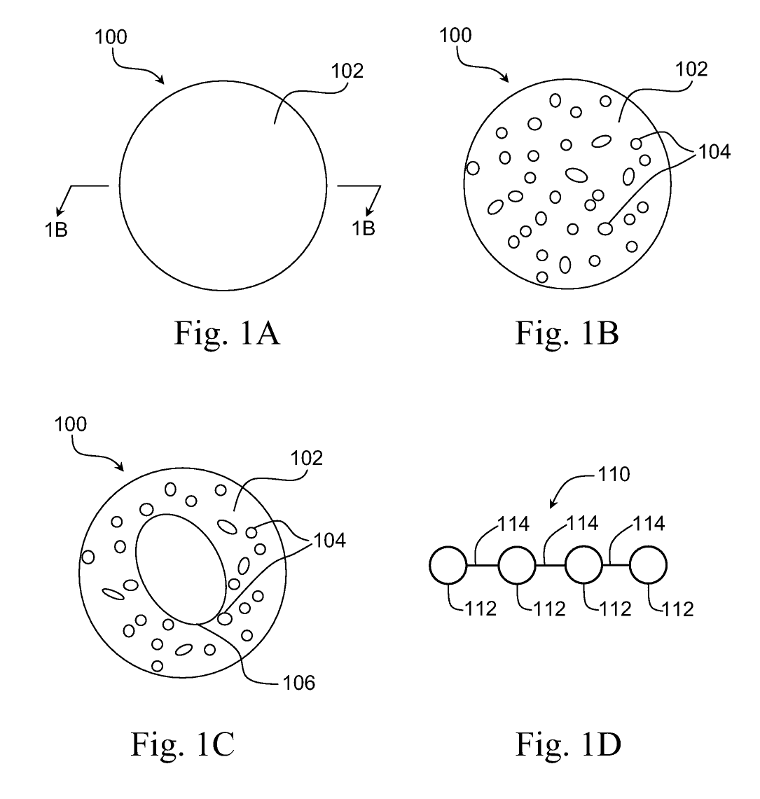

FIGS. 1A, 1B, and 1C show a particle 100 that can be used, for example, to deliver one or more therapeutic agents to a target site within the body. The particle 100 is formed from a polymer matrix 102 that is formed into the shape of a particle 100. The polymer matrix 102 can optionally include pores 104. The particle also can optionally include a cavity 106 surrounded by the polymer matrix 102. Therapeutic agent(s) can be included on and/or within particle 100 (e.g., within the polymer matrix 102, within the pores 104, within the cavity 106) and/or on the surface of particle 100.

The polymer matrix 102 of the particle 100 includes two polymers, i.e., a first polymer and a second polymer. The first polymer is one of polyvinyl alcohol or polyvinyl formal. The second polymer is one of polyvinyl alcohol, polyvinyl formal, polyvinylpyrrolidone, polysaccharide, or polymethacrylate. The first polymer and the second polymer are different. For example, if the first polymer is polyvinyl alcohol, the second polymer is not polyvinyl alcohol, but rather one of the other polymers listed above for the second polymer. The percent by weight of the first polymer in the polymer matrix 102 when compared to the weight of the second polymer is up to 99.99 percent or less (e.g., from 1 percent to 99 percent, from 5 percent to 95 percent, from 10 percent to 90 percent, from 20 percent to 80 percent, from 30 percent to 70 percent, from 40 percent to 60 percent, from 45 percent to 55 percent). In some embodiments, the percent by weight of the first polymer in the polymer matrix 102 when compared to the weight of the second polymer can be greater than 50 percent (e.g., greater than 55 percent, greater than 60 percent, greater than 65 percent, greater than 70 percent, greater than 75 percent, greater than 80 percent, greater than 85 percent), greater than 90 percent (e.g., greater than 91 percent, greater than 92 percent, greater than 94 percent, greater than 93 percent, greater than 95 percent, greater than 96 percent, greater than 97 percent, greater than 98 percent), or greater than 99 percent (e.g., greater than 99.1 percent, greater than 99.2 percent, greater than 99.3 percent, greater than 99.4 percent, greater than 99.5 percent, greater than 99.6 percent, greater than 99.7 percent, greater than 99.8 percent, or greater than 99.9 percent).

In some embodiments, the first polymer and the second polymer can be intimately mixed. The term “intimately mixed” as used herein refers to the polymers being well distributed within a mixture, e.g., the first polymer being well dispersed within the second polymer or vice versa. In other embodiments, discrete pockets of one type of polymer can exist inside another polymer.

In some embodiments, a third polymer can be included in the polymer matrix 102. The third polymer is one of polyvinyl alcohol, polyvinyl formal, polyvinylpyrrolidone, a polysaccharide, or a polymethacrylate. In these embodiments, the first polymer, second polymer, and third polymer are different. The percent by weight of the first polymer in the polymer matrix 102 including a third polymer is up to 99.99 percent or less (e.g., from 1 percent to 99 percent, from 5 percent to 95 percent, from 10 percent to 90 percent, from 20 percent to 80 percent, from 30 percent to 70 percent, from 40 percent to 60 percent, from 45 percent to 55 percent) when compared to the sum of the weights of the second polymer and third polymer. In some embodiments, the percent by weight of the first polymer in the polymer matrix 102 when compared to the sum of the weights of the second polymer and the third polymer can be greater than 50 percent (e.g., greater than 55 percent, greater than 60 percent, greater than 65 percent, greater than 70 percent, greater than 75 percent, greater than 80 percent, greater than 85 percent), greater than 90 percent (e.g., greater than 91 percent, greater than 92 percent, greater than 94 percent, greater than 93 percent, greater than 95 percent, greater than 96 percent, greater than 97 percent, greater than 98 percent), or greater than 99 percent (e.g., greater than 99.1 percent, greater than 99.2 percent, greater than 99.3 percent, greater than 99.4 percent, greater than 99.5 percent, greater than 99.6 percent, greater than 99.7 percent, greater than 99.8 percent, or greater than 99.9 percent).

Polyvinyl alcohol is useful in the compositions and particles described herein. For example, the matrix 102 of the particle 100 can include polyvinyl alcohol. As referred to herein, a vinyl alcohol monomer unit has the following structure:

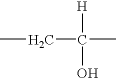

Polyvinyl alcohol is typically formed by partial or complete hydrolysis of polyvinyl acetate (to remove acetate groups). Hydrolysis of polyvinyl acetate is the typical route used to form polyvinyl alcohol because polyvinyl alcohol monomers almost exclusively exist in their tautomeric form, acetaldehyde. The percent hydrolysis of polyvinyl alcohol useful with the compositions described herein is 100 percent or less (e.g., from 1 percent to 99 percent, from 5 percent to 95 percent, from 10 percent to 90 percent, from 20 percent to 80 percent, from 30 percent to 70 percent, from 40 percent to 60 percent, from 45 percent to 55 percent). In some embodiments, the percent hydrolysis of polyvinyl alcohol is greater than 50 percent (e.g., greater than 55 percent, greater than 60 percent, greater than 65 percent, greater than 70 percent, greater than 75 percent, greater than 80 percent, greater than 85 percent), greater than 90 percent (e.g., greater than 91 percent, greater than 92 percent, greater than 94 percent, greater than 93 percent, greater than 95 percent, greater than 96 percent, greater than 97 percent, greater than 98 percent), or greater than 99 percent (e.g., greater than 99.1 percent, greater than 99.2 percent, greater than 99.3 percent, greater than 99.4 percent, greater than 99.5 percent, greater than 99.6 percent, greater than 99.7 percent, greater than 99.8 percent, or greater than 99.9 percent).

Polyvinyl formal is useful in the compositions and particles described herein. For example, the matrix 102 of the particle 100 can include a polymer including one or more vinyl formal monomer units, i.e., polyvinyl formal. As referred to herein, a vinyl formal monomer unit has the following structure:

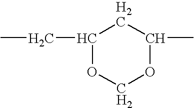

In certain embodiments, in addition to including one or more vinyl formal monomer units, polyvinyl formal can also include one or more vinyl alcohol monomer units (vinyl alcohol monomer units are described above). In some embodiments, in addition to including one or more vinyl formal monomer units and/or one or more vinyl alcohol monomer units, polyvinyl formal can also include one or more vinyl acetate monomer units. As referred to herein, a vinyl acetate monomer unit has the following structure:

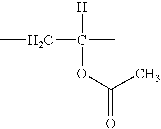

In embodiments in which the polyvinyl formal includes one or more vinyl formal monomer units, one or more vinyl alcohol monomer units, and/or one or more vinyl acetate monomer units, the monomer units generally can be arranged in a variety of different ways. As an example, in some embodiments, the polymer can include different monomer units that alternate with each other. For example, the polymer can include repeating blocks, each block including a vinyl formal monomer unit, a vinyl alcohol monomer unit, and a vinyl acetate monomer unit. As another example, in certain embodiments, the polymer can include blocks including multiple monomer units of the same type. For example, the polymer can include a block that is formed of multiple vinyl alcohol monomer units, connected to a block that is formed of multiple vinyl formal monomer units.

In some embodiments, polyvinyl formal can have the formula that is schematically represented below, in which x, y, and z each are integers that are greater than zero. The individual monomer units that are shown can be directly attached to each other, and/or can include one or more other monomer units (e.g., vinyl formal monomer units, vinyl alcohol monomer units, vinyl acetate monomer units) between them:

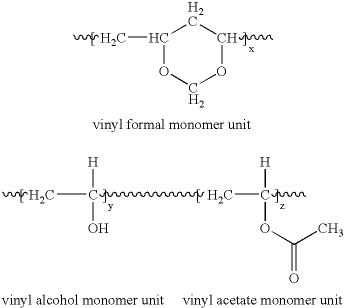

Examples of commercially available polymers including vinyl formal monomer units include the Vinylec® (formerly known as Formvar®) resins, available from SPI Supplies® (West Chester, Pa.). Vinylec® is a registered trade name for a family of copolymers including vinyl formal monomer units, vinyl alcohol monomer units, and vinyl acetate monomer units. A Vinylec® polymer includes 81 percent by weight vinyl formal monomer units, from 9.5 percent by weight to 13.0 percent by weight vinyl acetate monomer units, and from 5.0 percent by weight to 6.5 percent by weight vinyl alcohol monomer units. Different grades of Vinylec® polymers include Vinylec® E (previously Formvar® 15/95E), Vinylec® H (previously Formvar® 7/95E), Vinylec® L (previously Formvar® 6/95E), and Vinylec® K (previously Formvar® 5/95E).

Typically, as the weight percent of vinyl formal monomer units in a polymer increases, the hydrophobicity of a particle that is formed of the polymer can also increase. As the hydrophobicity of a particle increases, the particle can exhibit an enhanced ability to incorporate a hydrophobic therapeutic agent. As a result, the particle may be able to incorporate and/or deliver a relatively high volume of hydrophobic therapeutic agents. In certain embodiments, the weight percent of vinyl formal monomer units in a polymer used to form a particle can increase when the polymer is formalized prior to particle formation (e.g., while the polymer is in the solution state), rather than during and/or after particle formation. Without wishing to be bound by theory, it is believed that a polymer that is formalized prior to particle formation can include a higher number of polymer chains that are exposed to formalizing reactants during the formalization process, as compared to a polymer that is formalized during and/or after incorporation of the polymer into a particle.

In some embodiments, the polyvinyl formal can include at least 60 percent by weight (e.g., at least 65 percent by weight, at least 70 percent by weight, at least 80 percent by weight, at least 85 percent by weight, at least 90 percent by weight, at least 95 percent by weight), and/or at most 100 percent by weight (e.g., at most 95 percent by weight, at most 90 percent by weight, at most 85 percent by weight, at most 80 percent by weight, at most 75 percent by weight, at most 70 percent by weight, at most 65 percent by weight) vinyl formal monomer units. In certain embodiments, the polyvinyl formal can include more than 75 percent by weight vinyl formal monomer units. As used herein, the weight percent of vinyl formal monomer units in a polymer is measured using solid-state NMR spectroscopy, such as solid-state ¹³C NMR spectroscopy employing variable amplitude cross-polarization with high-power proton decoupling and magnetic angle spinning (VACP-MAS).

In some embodiments, a polymer including one or more vinyl formal monomer units can be formed using the following 1,3-acetalization process. As shown below, a section of a polymer including two vinyl alcohol monomer units is reacted with formaldehyde in the presence of an acid (e.g., sulfuric acid, hydrochloric acid, nitric acid, acetic acid, formic acid, phosphoric acid) to form water and a section of a polymer including one vinyl formal monomer unit:

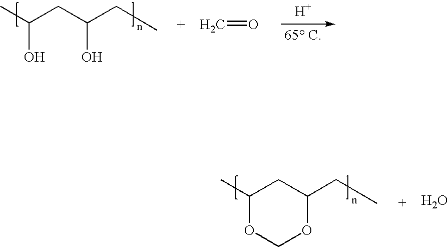

In embodiments in which the above process is used to form polyvinyl formal, the polymer can be substantially devoid of vinyl acetate monomer units (e.g., the polymer can contain less than 0.1 percent by weight vinyl acetate monomer units).

In certain embodiments in which the above process is used to form polyvinyl formal, some hydroxyl groups may not react with adjacent groups and may remain unconverted.

In some embodiments, an embodiment of a polymer including one or more vinyl formal monomer units can be formed by the following mechanism, in which n, x, y, and z each are integers that are greater than zero:

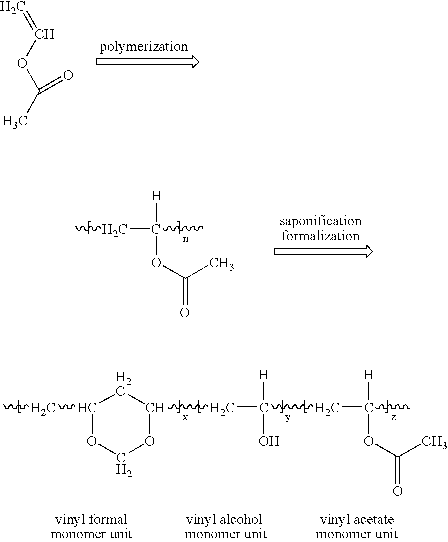

Polyvinylpyrrolidone is useful in the compositions described herein. For example, the matrix 102 of the particle 100 can include polyvinylpyrrolidone. As referred to herein, polyvinylpyrrolidone has the following structure:

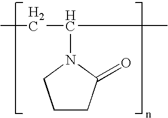

Additionally, the second or third polymer can be another polymer such as pyran copolymer, polyhydroxypropylmethacrylamide-phenol, polyhydroxyethylaspartamidephenol, or polyethyleneoxidepolylysine substituted with palmitoyl residues. The choice of the second or third polymer will depend on the drug release profile for the selected polymer. A polymer's drug release profile is related to its intrinsic properties, such as, chemical structure, e.g., the extent of cross-linking, or the presence of ionic groups;

Pharmaceutically acceptable polysaccharides are useful in the compositions described herein. Examples of polysaccharides useful in the polymer matrix described herein include, but are not limited to, methylcellulose, hydroxycellulose, hydroxy propylcellulose, hydroxy propylmethylcellulose, noncrystalline cellulose, polysaccharides including starch and starch derivatives such as hydroxyethylstarch (HES).

Pharmaceutically acceptable polymethacrylates are useful in the compositions described herein. Examples of polymethacrylates useful in the polymer matrix described herein include, but are not limited to, polymethacrylate, acrylic acid/methacrylic acid copolymers, methyl methacrylate copolymers, ethoxyethyl methacrylates, cyanoethyl methacrylate, poly(methacrylic acid), poly(methacrylic acid anhydride), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), aminoalkyl methacrylate copolymer, poly (trimethyl ammonioethyl methacrylate), and glycidyl methacrylate copolymers. Examples of glycidyl methacrylate copolymers include the EUDRAGIT®s (Rohm GmbH & Co. KG; Darmstadt, Germany) such as EUDRAGIT® RS-100, RL-100, RS-30D, RL-30D, RL-PO, RS-PO (ethyl acrylate methyl methacrylate trimethyl chloride methacrylate ammonium ethyl copolymer) and EUDRAGIT NE-30D (methyl methacrylate ethyl acrylate copolymer).

In general, the largest dimension of a particle as defined herein is 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the largest dimension of a particle is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the largest dimension of a particle is less than 100 microns (e.g., less than 50 microns).

In some embodiments, a particle described herein can be spherical or substantially spherical. In certain embodiments, a particle can have a sphericity of 0.8 or more (e.g., 0.85 or more, 0.9 or more, 0.95 or more, 0.97 or more). For embodiments in which a particle is compressible, the particle can be, for example, manually compressed (flattened) while wet to 50 percent or less of its original largest dimension and then, upon exposure to fluid, regain a sphericity of 0.8 or more (e.g., 0.85 or more, 0.9 or more, 0.95 or more, 0.97 or more). The sphericity of a particle can be determined using a Beckman Coulter RapidVUE Image Analyzer version 2.06 (Beckman Coulter, Miami, Fla.). Briefly, the RapidVuE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=√(4A/π); Dp=P/π; A 32 pixel area; P=pixel perimeter), is a value from zero to one, with one representing a perfect circle.

In some embodiments, two or more particles can be linked together to form a particle chain 110 as shown in FIG. 1D, e.g., a particle portion 112 of the particle chain 110 can be connected by a linkage portion 114 to at least one other particle portion 112. The particle portions 112 can be connected to each other in the particle chain 110 by linkage portions 114 that are formed of one or more of the same material(s) as the particle portions 112, or of one or more different material(s) from the particle portions 112. For example, the linkage portions 114 can be formed from a polymer, a metal, or a fiber. Additionally, a particle portion 112 can be connected to a particle or particles dissimilar to particle portion 112.

In general, a particle portion 112 can have a largest dimension of 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the largest dimension of particle portion 112 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the largest dimension of particle portion 112 is less than 100 microns (e.g., less than 50 microns).

In some embodiments, a particle portion 112 can be substantially spherical. In certain embodiments, a particle portion 112 can have a sphericity of 0.8 or more (e.g., 0.85 or more, 0.9 or more, 0.95 or more, 0.97 or more). In some embodiments, the particle portion 112 is compressible. The particle portion 112 can be, for example, manually compressed, essentially flattened, while wet to 50 percent or less of its original largest dimension and then, upon exposure to fluid, regain a sphericity of 0.8 or more (e.g., 0.85 or more, 0.9 or more, 0.95 or more, 0.97 or more). The sphericity of a particle can be determined using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=√(4A/π); Dp=P/π; A=pixel area; P=pixel perimeter), is a value from zero to one, with one representing a perfect circle.

In general, the particle chain 110 can have a restrained length of from one centimeter to 50 centimeters. The restrained length LR of the particle chain 110 is the maximum length of the particle chain 110 (the length of the particle chain 110 when the particle chain 110 is taut) in any dimension. In some embodiments, the particle chain 110 can have a restrained length of at least one centimeter (e.g., at least five centimeters, at least ten centimeters, at least 15 centimeters, at least 20 centimeters, at least 25 centimeters, at least 30 centimeters, at least 35 centimeters, at least 40 centimeters, at least 45 centimeters) and/or at most 50 centimeters (e.g., at most 45 centimeters, at most 40 centimeters, at most 35 centimeters, at most 30 centimeters, at most 25 centimeters, at most 20 centimeters, at most 15 centimeters, at most ten centimeters, at most five centimeters).

The particle chain 110 includes at least two particle portions 112 (e.g., from two particle portions to 1,000 particle portions). In some embodiments, the particle chain 110 can include at least two particle portions 112 (e.g., at least five particle portions; at least ten particle portions; at least 20 particle portions; at least 30 particle portions; at least 40 particle portions; at least 50 particle portions; at least 100 particle portions; at least 250 particle portions; at least 500 particle portions; at least 750 particle portions; at least 1,000 particle portions; at least 2,500 particle portions) and/or at most 5,000 particle portions (e.g., at most 2,500 particle portions; at most 1,000 particle portions; at most 750 particle portions; at most 500 particle portions; at most 250 particle portions; at most 100 particle portions; at most 50 particle portions; at most 40 particle portions; at most 30 particle portions; at most 20 particle portions; at most ten particle portions; at most five particle portions). For example, the particle chain 110 can include five particle portions, ten particle portions, 100 particle portions, 500 particle portions, or 1,000 particle portions.

The particle portions 112 in the particle chain 110 can all have approximately the same largest dimension or can have different largest dimension. As an example, in some embodiments, the particle portions 112 at one end of the particle chain 110 can have a larger largest dimension (e.g., by 1100 microns) than the particle portions 112 at the other end of the particle chain 110. As another example, in certain embodiments, the particle portions 112 in the particle chain 110 can alternate in size. For example, a particle portion 112 with a largest dimension of 300 microns can be adjacent to a particle portion 112 with a largest dimension of 500 microns.

The linkage portions 114 generally can have a width of from 0.001 inch to 0.01 inch (e.g., from 0.003 inch to 0.005 inch). In certain embodiments, the linkage portions 114 can have a width of at least 0.001 inch (e.g., at least 0.002 inch, at least 0.003 inch, at least 0.004 inch, at least 0.005 inch, at least 0.006 inch, at least 0.007 inch, at least 0.008 inch, at least 0.009 inch) and/or at most 0.01 inch (e.g., at most 0.009 inch, at most 0.008 inch, at most 0.007 inch, at most 0.006 inch, at most 0.005 inch, at most 0.004 inch, at most 0.003 inch, at most 0.002 inch).

In some embodiments, the linkage portions 114 in the particle portion 112 can all have approximately the same length and/or width. In other embodiments, the particle portion 112 can include linkage portions 114 of varying lengths and/or widths. As an example, in certain embodiments, one end of a particle chain 110 can have relatively short, thick links, while the other end of the particle chain 110 has relatively long, thin links. As another example, in some embodiments, the linkage portions 114 in a particle chain 110 can alternate between being relatively short and thick and relatively long and thin.

In general, the linkage portions 114 can have an aspect ratio (the ratio of the length of the link to the width of the link) of from zero to 1,000. In some embodiments, the linkage portions 114 can have an aspect ratio of at least 0.001 (e.g., at least 0.005, at least 0.5, at least one, at least five, at least ten, at least 15, at least 20, at least 25, at least 26, at least 30, at least 40, at least 50, at least 75, at least 100, at least 200, at least 300, at least 400, at least 500, at least 600, at least 700, at least 800, at least 900 ) and/or at most 1,000 (e.g., at most 900, at most 800, at most 700, at most 600, at most 500, at most 400, at most 300, at most 200, at most 100, at most 75, at most 50, at most 40, at most 30, at most 26, at most 25, at most 20, at most 15, at most ten, at most five, at most one, at most 0.5, at most 0.005).

In general, the aspect ratio of the linkage portions 114 can be varied as desired. Typically, as the aspect ratio of the linkage portions 114 increases, the flexibility of the linkage portions 114 increases. As the aspect ratio of the linkage portions 114 decreases, the tensile strength of the linkage portions 114 typically increases.

In some embodiments, the ratio of the largest dimension of a particle portion 112 to the width of a linkage portions 114 can be from 0.5 to 100. The ratio can be at least 0.5 (e.g., at least 0.8, at least one, at least two, at least five, at least ten, at least 12, at least 15, at least 20, at least 25, at least 30, at least 35, at least 40, at least 45, at least 50, at least 55, at least 60, at least 70. at least 80, at least 90) and/or at most 100 (e.g., at most 90, at most 80, at most 70, at most 60, at most 55, at most 50, at most 45, at most 40, at most 35, at most 30, at most 25, at most 20, at most 15, at most 12, at most ten, at most five, at most two, at most one, at most 0.8).

Generally, the ratio of the largest dimension of a particle portion 112 to the width of a linkage portions 114 can be varied as desired. Typically, as the ratio of the largest dimension of a particle portion 112 to the width of a linkage portions 114 increases, the flexibility of the linkage portions 114 increases. As the ratio of the largest dimension of a particle portion 112 to the width of a linkage portion decreases, the tensile strength of the linkage portions 114 typically increases.

Polymers useful in the linkage portion 114 described herein include homopolymers and copolymers. The term “homopolymer” as used herein refers to a polymer formed from identical monomer subunits. Examples of homopolymers useful in the particles described herein include, but are not limited to, polyvinyl alcohols (“PVA”), polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polyolefins, polypropylenes, polymethylmethacrylates, polycaprolactones, polyglycolic acids, poly(lactic-co-glycolic) acids (e.g., poly(d-lactic-co-glycolic) acids), polysulfones, polyethersulfones, polycarbonates, nylons, silicones, and linear or crosslinked polysilicones. Copolymers useful with the particles described herein can be formed from combinations of the monomers that make up these homopolymers.

Particle chains and their characteristics are further described, for example, in Buiser et al., U.S. Patent Application Publication No. US 2005/0238870 A1, published on Oct. 27, 2005, and entitled “Embolization,” which is incorporated herein by reference.

As noted above and shown in FIG. 1, particle 100 can include a cavity 106 surrounded by the polymer matrix 102 including pores 104. For a given particle containing a cavity 106, the cavity occupies at least 30 percent by volume of the particle, and a pore occupies less than one percent by volume of the particle.

The presence of cavity 106 and pores 104 in particle 100 can enhance the ability of particle 100 to retain and/or deliver a relatively large volume of therapeutic agent. As an example, in some embodiments, cavity 106 can be used to store a relatively large volume of therapeutic agent, and/or pores 104 can be used to deliver the relatively large volume of therapeutic agent into a target site within a body of a subject at a controlled rate. As another example, in certain embodiments, both cavity 106 and pores 104 can be used to store and/or deliver one or more therapeutic agents. In some embodiments, cavity 106 can contain one type of therapeutic agent, while pores 104 contain a different type of therapeutic agent.

Generally, as the size of a cavity 106 in a particle 100 increases, the volume of therapeutic agent retained by particle 100 can increase. In some embodiments, cavity 106 can have a largest dimension of at least one micron (e.g., a least five microns, at least 10 microns, at least 25 microns, at least 50 microns, at least 100 microns, at least 250 microns, at least 500 microns, at least 750 microns) and/or at most 1,000 microns (e.g., at most 750 microns, at most 500 microns, at most 250 microns, at most 100 microns, at most 50 microns, at most 25 microns, at most 10 microns, at most five microns). While particle 100 is shown as having one cavity 106, in certain embodiments, a particle can include more than one cavity (e.g., two cavities, three cavities, four cavities, or five cavities).

Typically, as the sizes of pores 104 increase, the volume of therapeutic agent retained by a particle 100 can increase. In certain embodiments, one or more pores 104 can have a largest dimension of at least 0.01 micron (e.g., at least 0.05 micron, at least 0.1 micron, at least 0.5 micron, at least one micron, at least five microns, at least 10 microns, at least 15 microns, at least 20 microns, at least 25 microns, at least 30 microns, at least 35 microns, at least 50 microns, at least 100 microns, at least 150 microns, at least 200 microns, at least 250 microns), and/or at most 300 microns (e.g., at most 250 microns, at most 200 microns, at most 150 microns, at most 100 microns, at most 50 microns, at most 35 microns, at most 30 microns, at most 25 microns, at most 20 microns, at most 15 microns, at most 10 microns, at most five microns, at most one micron, at most 0.5 micron, at most 0.1 micron, at most 0.05 micron). In some embodiments, some or all of pores 104 can have a maximum dimension of from 0.01 micron to one micron, and/or some or all of pores 104 can have a maximum dimension of from 10 microns to 300 microns (e.g., from 100 microns to 300 microns). As used herein, pore size is measured using mercury porosimetry.

While FIGS. 1B and 1C show a particle with pores having different sizes, in some embodiments, a particle can include pores that have the same size (e.g., that have the same largest dimension).

As described above, the particle 100 can be used to deliver one or more therapeutic agents (e.g., a combination of therapeutic agents) to a target site. Therapeutic agents include genetic therapeutic agents, non-genetic therapeutic agents, and cells, and can be negatively charged, positively charged, amphoteric, neutral, hydrophilic, or hydrophobic. Therapeutic agents can be, for example, materials that are biologically active to treat physiological conditions; pharmaceutically active compounds; proteins; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Examples of radioactive species include yttrium (⁹⁰Y), holmium (¹⁶⁶Ho), phosphorus (³²P), (¹⁷⁷Lu), actinium (²²⁵Ac), praseodymium, astatine (²¹¹At), rhenium (¹⁸⁶Re), bismuth (²¹²Bi or ²¹³Bi),), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), sulfur (³⁵S), carbon (¹⁴C), tritium (³H), chromium (⁵¹Cr), chlorine (³⁶Cl), cobalt (⁵⁷Co or ⁵⁸Co), iron (59Fe), selenium (⁷⁵Se), and/or gallium (⁶⁷Ga). In some embodiments, yttrium (⁹⁰Y), lutetium (¹⁷⁷Lu), actinium (²²⁵Ac), praseodymium, astatine (211At), rhenium (186Re), bismuth (²¹²Bi or ²¹³Bi), holmium (¹⁶⁶Ho), samarium (¹⁵³Sm), iridium (¹⁹² Ir), and/or rhodium (¹⁰⁵Rh) can be used as therapeutic agents. In certain embodiments, yttrium (⁹⁰Y), lutetium (¹⁷⁷Lu), actinium (²²⁵ Ac), praseodymium, astatine (²¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium (¹⁰⁵Rh), iodine (131I or ¹²⁵I), indium (¹¹¹In), technetium (⁹⁹Tc), phosphorus (³²P), carbon (¹⁴C), and/or tritium (³H) can be used as a radioactive label (e.g., for use in diagnostics). In some embodiments, a radioactive species can be a radioactive molecule that includes antibodies containing one or more radioisotopes, for example, a radiolabeled antibody. Radioisotopes that can be bound to antibodies include, for example, iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁰⁵Rh), sulfur (³⁵S), carbon (14C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). Examples of antibodies include monoclonal and polyclonal antibodies including RS7, Mov18, MN-14 IgG, CC49, COL-1, mAB A33, NP-4 F(ab′)2 anti-CEA, anti-PSMA, ChL6, m-170, or antibodies to CD20, CD74 or CD52 antigens. Examples of radioisotope/antibody pairs include m-170 MAB with ⁹⁰Y. Examples of commercially available radioisotope/antibody pairs include Zevalin™ (Biogen IDEC; San Diego, Calif.) and Bexxar™ (Corixa Corporation, Seattle, Wash.). Further examples of radioisotope/antibody pairs can be found in J. Nucl. Med. Arpil 2003,: 44(4): 632-40.

Non-limiting examples of therapeutic agents include anti-thrombogenic agents; thrombogenic agents; agents that promote clotting; agents that inhibit clotting; antioxidants; angiogenic and anti-angiogenic agents and factors; anti-proliferative agents (e.g., agents capable of blocking smooth muscle cell proliferation, such as rapamycin); calcium entry blockers (e.g., verapamil, diltiazem, nifedipine); targeting factors (e.g., polysaccharides, carbohydrates); agents that can stick to the vasculature (e.g., charged moieties, such as gelatin, chitosan, and collagen); and survival genes which protect against cell death (e.g., anti-apoptotic Bcl-2 family factors and Akt kinase).

Examples of non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Examples of genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them.

Vectors of interest for delivery of genetic therapeutic agents include: plasmids; viral vectors such as adenovirus (AV), adenoassociated virus (AAV) and lentivirus; and non-viral vectors such as lipids, liposomes, and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents are disclosed in Kunz et al., U.S. Pat. No. 5,733,925, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

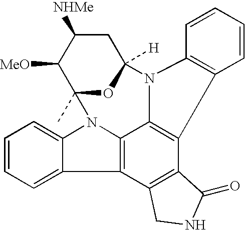

as well as diindoloalkaloids having one of the following general structures:

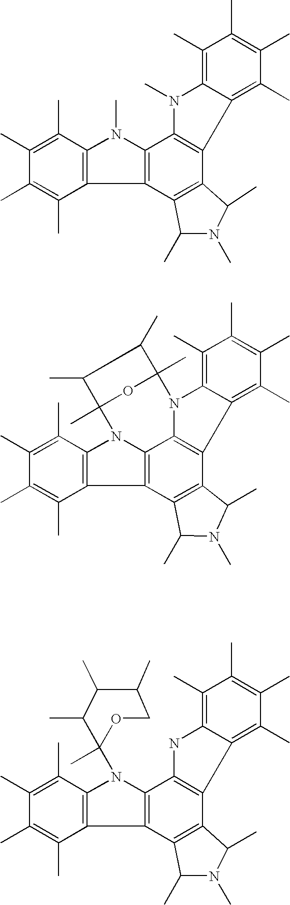

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i.e., those that have only a central sesquiterpenoid structure) and macrocyclic trichothecenes (i.e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including α-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP Iib/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), Fxa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE1 and PGI2; prostacyclins and prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganic ions (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, interleukins, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

In some embodiments, a therapeutic agent can be hydrophilic. An example of a hydrophilic therapeutic agent is doxorubicin hydrochloride. In certain embodiments, a therapeutic agent can be hydrophobic. Examples of hydrophobic therapeutic agents include paclitaxel, cisplatin, tamoxifen, and doxorubicin base. In some embodiments, a therapeutic agent can be lipophilic. Examples of lipophilic therapeutic agents include taxane derivatives (e.g., paclitaxel) and steroidal materials (e.g., dexamethasone).

Therapeutic agents as used herein can be combined with pharmaceutically acceptable carriers including solvents. As used herein, the term “carrier” encompasses any excipient, diluent, filler, salt, buffer, stabilizer, solubilizer, lipid, stabilizer, or other material well known in the art for use in pharmaceutical formulations. The choice of a carrier will depend upon the therapeutic agent. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005.

Examples of physiologically acceptable carriers include buffers such as phosphate buffers, citrate buffer, and buffers with other organic acids; antioxidants including ascorbic acid; low molecular weight (10 or fewer residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.). Examples of suitable aqueous and nonaqueous carriers, diluents, solvents or vehicles include water, glacial acetic acid, ethanol, polyols (propyleneglycol, polyethyleneglycol, glycerol, and the like), suitable mixtures thereof, vegetable oils (such as olive oil) and injectable organic esters such as ethyl oleate.

Therapeutic agents are described, for example, in DiMatteo et al., U.S. Patent Application Publication No. US 2004/0076582 Al, published on Apr. 22, 2004, and entitled “Agent Delivery Particle”; Schwarz et al., U.S. Pat. No. 6,368,658; Buiser et al., U.S. patent application Ser. No. 11/311,617, filed on Dec. 19, 2005, and entitled “Coils”; and Song, U.S. patent application Ser. No. 11/355,301, filed on Feb. 15, 2006, and entitled “Block Copolymer Particles”, all of which are incorporated herein by reference. In certain embodiments, in addition to or as an alternative to including therapeutic agents, the particle 100 can include one or more radiopaque materials, materials that are visible by magnetic resonance imaging (MRI-visible materials), ferromagnetic materials, and/or contrast agents (e.g., ultrasound contrast agents). Radiopaque materials, MRI-visible materials, ferromagnetic materials, and contrast agents are described, for example, in Rioux et al., U.S. Patent Application Publication No. US 2004/0101564 A1, published on May 27, 2004, and entitled “Embolization”, which is incorporated herein by reference.

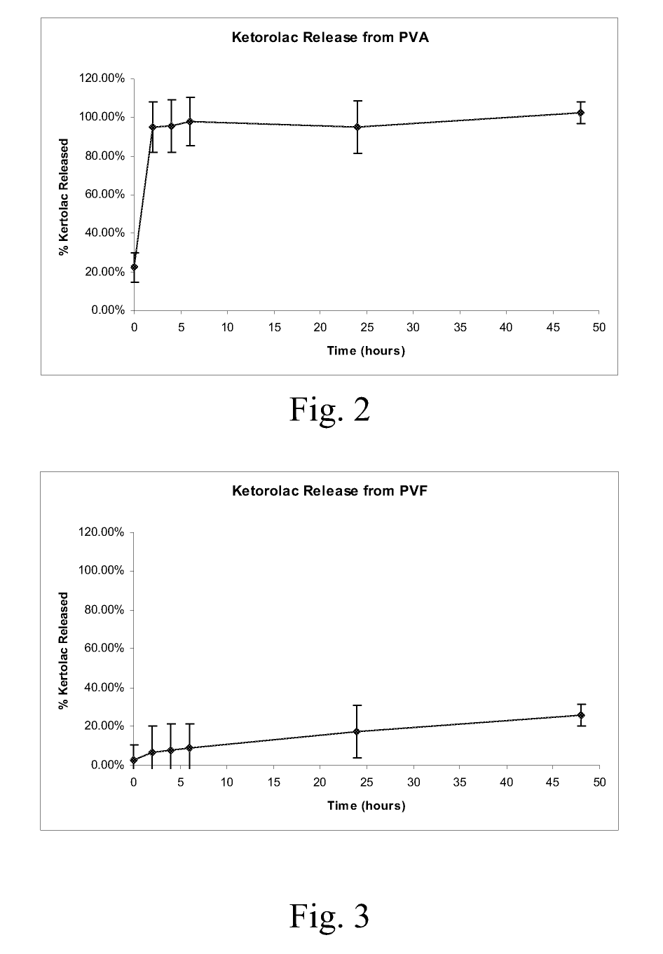

The compositions as described herein can be formulated such that the rate of release of a therapeutic agent from the composition is controlled. For example, as shown in FIG. 2 (see description at Example 1 below), polyvinyl alcohol alone can release 95 percent to 100 percent of a therapeutic agent, in this case ketorolac tromethamine, in two hours, followed by uniform release of the remaining agent within 48 hours. However, a composition containing polyvinyl alcohol and polyvinyl formal can reduce the amount of therapeutic agent released in the first two hours and provide uniform release thereafter for an extended period of time. The ratio of the first polymer to the second polymer in the compositions described herein can provide different rates of release of a therapeutic agent from the composition. By changing the ratio of the first polymer to the second polymer a therapeutic agent release profile tailored to a specific purpose can be created. For example, a 50:50 combination of PVA and PVF should provide a controllec release of ketorolac of about 60% in 48 hours.

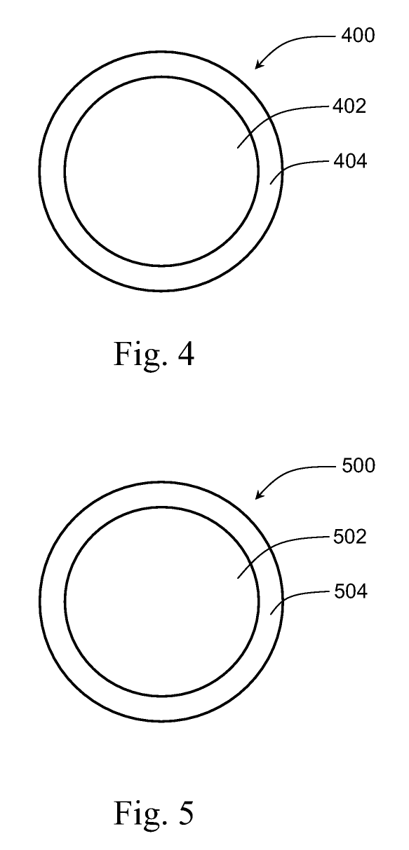

A desired therapeutic agent release profile can be a modification of a known therapeutic release profile such as, for example, the release profile of polyvinyl alcohol for ketorolac tromethamine as discussed above and shown in FIG. 2, or polyvinyl formal, which has a slow sustained release rate for ketorolac tromethamine, which is shown in FIG. 3 (see description at Example 1 below). For example, by adding polyvinylpyrrolidone to a polyvinyl alcohol composition, the amount of therapeutic agent released in the initial burst rate of polyvinyl alcohol can be reduced leaving additional therapeutic agent to be released over time at a sustained rate. Conversely, adding polyvinylpyrrolidone to a polyvinyl formal composition can provide an increased rate of release of a therapeutic agent while maintaining a desired amount of therapeutic agent for release over time at a sustained rate. The compositions described herein provide the ability to tailor the therapeutic agent release profiles of known polymers. Because different therapeutic agents may have different release profiles from a given polymer, different compositions may provide similar release profiles for different therapeutic agents.

In certain embodiments, the type of solvent used as a carrier for a therapeutic agent can also impact the release rate for the therapeutic agent. Without intending to be bound by theory, it is thought that the physical properties of a solvent such as dipole moment and hydrogen bonding potential impact the ability of a therapeutic agent to migrate from the interior of a composition matrix. For example, water can interact differently than glacial acetic acid in a matrix containing polyvinyl alcohol.

In other embodiments, other polymers, such as polyvinylpyrrolidone, polysaccharides, and polymethacrylates can be added to polyvinyl alcohol or polyvinyl formal to create different release profiles. Additionally, a third polymer can be added to a two polymer composition thereby further modifying the release profile for a therapeutic agent.

In certain embodiments a polymer composition with a specific release profile for a specific therapeutic agent can be generated. First, a desired release profile is determined. Then a polymer composition is created by combining a first polymer and a second polymer. The first polymer is one of polyvinyl alcohol or polyvinyl formal. The second polymer is one of polyvinyl alcohol, polyvinyl formal, polyvinylpyrrolidone, polysaccharide, or polymethacrylate. The first polymer and the second polymer are different. For example, if the first polymer is polyvinyl alcohol, the second polymer is not polyvinyl alcohol, but rather one of the other polymers listed above for the second polymer. Next the release profile of the specific therapeutic agent is determined. Finally, the determined release profile is compared with the desired release profile. If the determined release profile matches the desired release profile, or is otherwise acceptable, the desired composition has been established. If the determined release profile does not match the desired release profile or is otherwise unacceptable, another polymer composition is created and similarly analyzed and compared with the desired release profile. Composition modification can continue until either the determined release profile matches the desired release profile, or the determined release profile is acceptable.

In addition to particles, in certain embodiments the compositions as described herein can be used to form devices, such as devices for directed drug delivery, tubing, stents, and surgical devices. Methods for forming such devices are well known to those of skill in the art and include, but are not limited to, methods such as injection molding and extrusion.

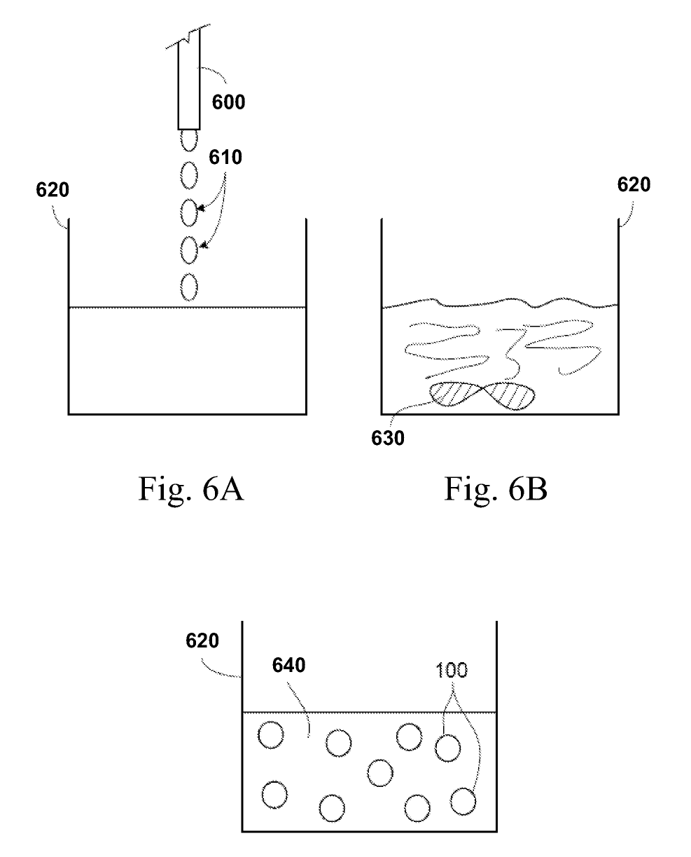

In other embodiments, the compositions as described herein can be used as a coating. For example, a particle can be coated with a composition as described herein. As shown in FIG. 4, particle 400 can include particle portion 402 and a coating portion 404, which is formed from a composition as discussed herein. Additionally, other medical devices such as tubing, stents, or surgical devices (e.g., a clamp) can be coated with a composition as described herein. Methods for coating are well known to those of skill in the art and include, but are not limited to, dip coating and spray coating.

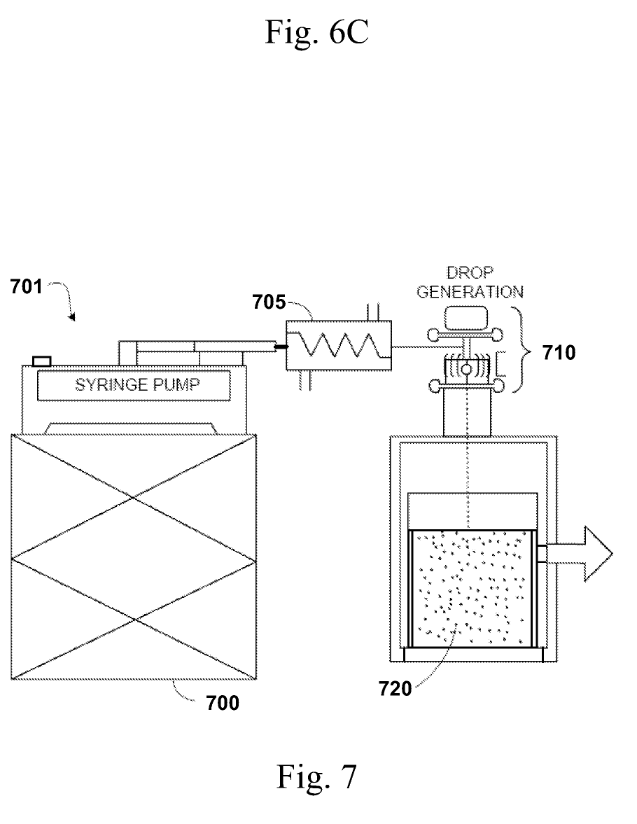

In certain embodiments, a particle, particle chain, or device as described above can also include a coating (or additional coating in the case of a particle, particle chain, or device that is coated with a composition as described herein). For example, FIG. 5 shows a particle 500 with an interior region 502 and a coating 504 formed of a polymer composition (e.g., polyvinyl alcohol) that is different from the polymer composition in the interior region 502. The interior region 502 is the polymer matrix discussed above. The size and other physical parameters of particle 500 are the same as those discussed above for particle 100. Coating 504 can, for example, regulate the release of therapeutic agent from particle 500, and/or can provide protection to the interior region 502 of the particle 500 (e.g., during delivery of particle 500 to a target site). In certain embodiments, coating 504 can be formed of a bioerodible and/or bioabsorbable material that can erode and/or be absorbed as particle 500 is delivered to a target site. This can, for example, allow interior region 502 to deliver a therapeutic agent to the target site once particle 500 has reached the target site. A bioerodible material can be, for example, a polysaccharide (e.g., alginate); a polysaccharide derivative; an inorganic, ionic salt; a water soluble polymer (e.g., polyvinyl alcohol, such as polyvinyl alcohol that has not been cross-linked); biodegradable poly DL-lactide-poly ethylene glycol (PELA); a hydrogel (e.g., polyacrylic acid, hyaluronic acid, gelatin, carboxymethyl cellulose); a polyethylene glycol (PEG); chitosan; a polyester (e.g., a polycaprolactone); a poly(ortho ester); a polyanhydride; a poly(lactic-co-glycolic) acid (e.g., a poly(d-lactic-co-glycolic) acid); a poly(lactic acid) (PLA); a poly(glycolic acid) (PGA); or a combination thereof. In some embodiments, coating 504 can be formed of a swellable material, such as a hydrogel (e.g., polyacrylamide co-acrylic acid). The swellable material can be made to swell by, for example, changes in pH, temperature, and/or salt. In certain embodiments in which particle 500 is used in an embolization procedure, coating 504 can swell at a target site, thereby enhancing occlusion of the target site by particle 500.

Particles can be formed by any of a number of different methods.

One method for making a particle involves combining a first polymer and a second polymer to form a polymer composition. The first polymer is one of polyvinyl alcohol or polyvinyl formal. The second polymer is one of polyvinyl alcohol, polyvinyl formal, polyvinylpyrrolidone, a polysaccharide, or a polymethacrylate. In this polymer composition, the first polymer and the second polymer are different. Once the polymers of the polymer composition are combined, a particle is formed from the polymer composition. A third polymer can optionally be combined with the first polymer and second polymer. The third polymer can be one of polyvinyl alcohol, polyvinyl formal, polyvinylpyrrolidone, a polysaccharide, or a polymethacrylate. In this three polymer composition, the first polymer, second polymer, and third polymer are different.

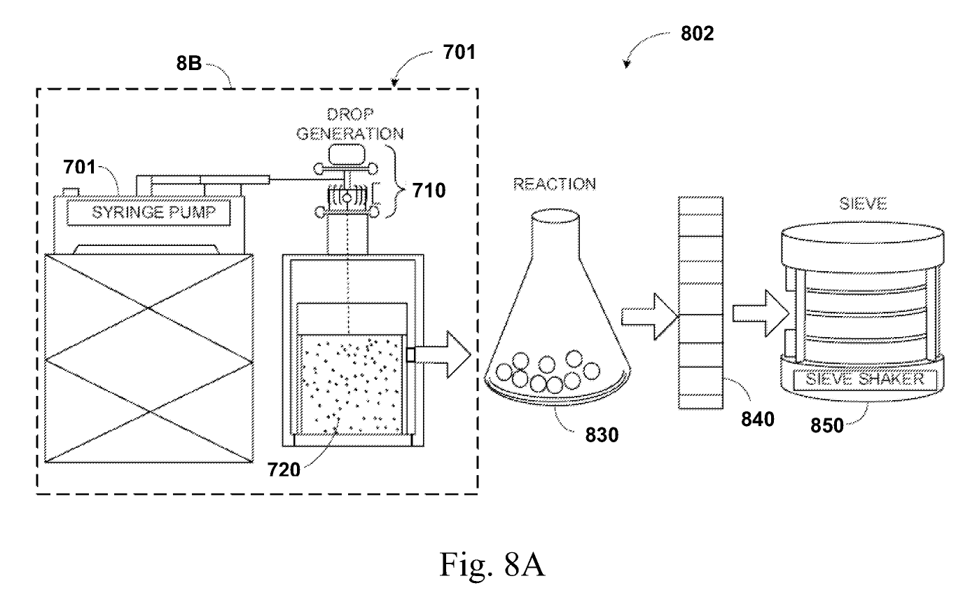

As an example of a method of forming particles as just described, FIGS. 6A-6C show a single-emulsion process that can be used, for example, to make particles such as particle 100 (FIGS. 1A, 1B, and 1C).

As shown in FIGS. 6A-6C, a drop generator 600 (e.g., a pipette, a needle) forms drops 610 of an organic solution including an organic solvent, a therapeutic agent, and the polymer matrix. Examples of organic solvents include glacial acetic acid, N,N-dimethylformamide (DMF), tetrahydrofuran (THF), and dimethylsulfoxide (DMSO). In certain embodiments, the organic solvent can be an aprotic polar solvent (e.g., DMF), which can dissolve both polar therapeutic agents and some non-polar therapeutic agents. In some embodiments, the organic solution can include at least 70 percent by volume (e.g., at least 80 percent by volume, at least 90 percent by volume) and/or at most 97 percent by volume (e.g., at most 90 percent by volume, at most 80 percent by volume) of the organic solvent. In certain embodiments, the organic solution can include at least three percent weight/volume (w/v) (e.g., at least 10 percent w/v, at least 20 percent w/v), and/or at most 30 percent w/v (e.g., at most 20 percent w/v, at most 10 percent w/v), of the polymer matrix. As an example, in some embodiments, the organic solution can include 10 percent w/v of the polymer matrix. In general, as the concentration of the polymer in the organic solution increases, the sizes and/or masses of the particles that are formed from the organic solution can also increase. Typically, as the volume of organic solvent in the organic solution that is used to form drops 610 decreases, the rate at which particles form can increase. Generally, the rate of particle formation can increase as the volume of organic solvent that is used decreases. Without wishing to be bound by theory, it is believed that this occurs because the organic solvent can evaporate from drops 610 more quickly during the particle formation process.

After they are formed, drops 610 fall from drop generator 600 into a vessel 620 that contains an aqueous solution including water (e.g., from 50 milliliters to 100 milliliters of water) and a surfactant. Examples of surfactants include lauryl sulfate, polyvinyl alcohols, poly(vinyl pyrrolidone) (PVP), and polysorbates (e.g., Tween® 20, Tween® 80). The concentration of the surfactant in the aqueous solution can be at least 0.1 percent w/v (e.g., at least 0.5 percent w/v, at least one percent w/v, at least three percent w/v), and/or at most five percent w/v (e.g., at most three percent w/v, at most one percent w/v, at most 0.5 percent w/v). For example, in some embodiments, the solution can include one percent w/v lauryl sulfate. Generally, as the concentration of the surfactant in the aqueous solution increases, the sphericity of the particles that are produced from the drop generation process and the rate of formation of the particles during the particle formation process, can also increase. In some embodiments, the aqueous solution can be at a temperature of at least 4° C. (e.g., at least 10° C., at least 20° C., at least 30° C.) and/or at most 40° C. (e.g., at most 30° C., at most 20° C., at most 10° C.). Typically, as the temperature of the aqueous solution increases, the rate at which particles (e.g., relatively rigid particles) form can also increase.

As FIG. 6B shows, after drops 610 have fallen into vessel 620, the solution is mixed (e.g., homogenized) using a stirrer 630. In some embodiments, the solution can be mixed for a period of at least six hours (e.g., at least 15 hours, at least 30 hours) and/or at most 48 hours (e.g., at most 30 hours, at most 15 hours). In certain embodiments, mixing can occur at a temperature of at least 4° C. (e.g., at least 10° C., at least 20° C., at least 30° C.) and/or at most 40° C. (e.g., at most 30° C., at most 20° C., at most 10° C.). The mixing results in a suspension 640 including particles 100 suspended in the solvent (FIG. 6C). Without wishing to be bound by theory, it is believed that the water insolubility of the polymer including vinyl formal monomer units can cause the polymer to form particles when the polymer is added into an aqueous solution and mixed.

After particles 100 have been formed, they are separated from the solvent by, for example, filtration (e.g., through a drop funnel, filter paper, and/or a wire mesh), centrifuging followed by removal of the supernatant, and/or decanting. Thereafter, particles 100 are dried (e.g., by evaporation, by vacuum drying, by air drying). In some embodiments, combinations of drying methods can be used. As an example, in certain embodiments, particles 100 can be air-dried. In certain embodiments, after being formed, particles 100 can be stored in a carrier fluid, such as saline. In some embodiments, particles 100 can be stored in deionized water.

In certain embodiments, the porosity of particles (e.g., particles 100) that are formed using an emulsion process (e.g., a single-emulsion process, such as the process describe above with reference to FIGS. 6A-6C) can be increased or decreased by adjusting one or more of the parameters of the emulsion process. As an example, as the viscosity of the organic solution increases, the porosity of the resulting particles can decrease. In some embodiments (e.g., embodiments in which the organic solution includes five percent w/v of the polymer including vinyl formal monomer units), the organic solution can have a viscosity of at least eight mPa (e.g., at least 20 mPa, at least 35 mPa) and/or at most 51.2 mPa (e.g., at most 35 mPa, at most 20 mPa). As used herein, the viscosity of an organic solution is measured at 20° C., using an Ostwald Viscometer. As another example, as the volume of organic solvent used in the organic solution decreases, the porosity of the resulting particles can decrease. Without wishing to be bound by theory, it is believed that when the organic solvent leaves the polymer phase during the particle formation process, it can cause pores to form in the resulting particles. Thus, when a smaller volume of organic solvent is used in the particle formation process, the particles that are formed may have a lower porosity. As an additional example, in some embodiments, as the volume of water in the aqueous solution increases, the porosity of the resulting particles can also increase. As a further example, as the weight per unit volume of surfactant in the aqueous solution increases, the porosity of the resulting particles can also increase.

In some embodiments, the size of drop generator 600 (e.g., the needle gauge, the pipette maximum dimension) can be selected to result in the formation of particles 100 of a desired size. Typically, as the size of drop generator 600 decreases, the size of particles 100 can also decrease.

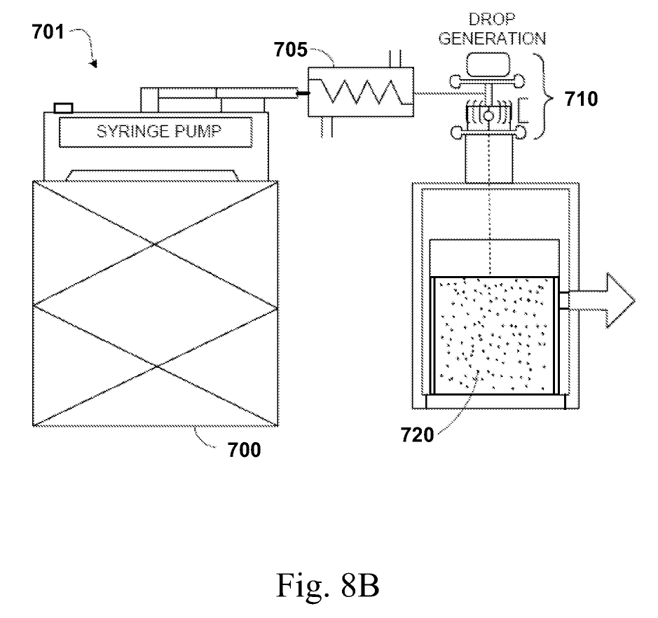

While pipettes and needles have been described as examples of drop generators that can be used in a particle formation process, in some embodiments, other types of drop generators or drop generator systems can be used in a particle formation process. For example, FIG. 7 shows a drop generator system 701 that includes a flow controller 700, a viscosity controller 705, a drop generator 710, and a vessel 720. Flow controller 700 delivers a solution (e.g., a solution including a solvent, a therapeutic agent, and a polymer matrix) to viscosity controller 705, which heats the solution to reduce its viscosity prior to delivery to drop generator 710. The solution then passes through an orifice in a nozzle in drop generator 710, resulting in the formation of drops of the solution. The drops are then directed into vessel 720, which contains, for example, an aqueous solution including a surfactant such as polyvinyl alcohol (PVA). Drop generators are described, for example, in Lanphere et al., U.S. Patent Application Publication No. US 2004/0096662 A1, published on May 20, 2004, and entitled “Embolization”, and in DiCarlo et al., U.S. patent application Ser. No. 11/111,511, filed on Apr. 21, 2005, and entitled “Particles”, both of which are incorporated herein by reference.

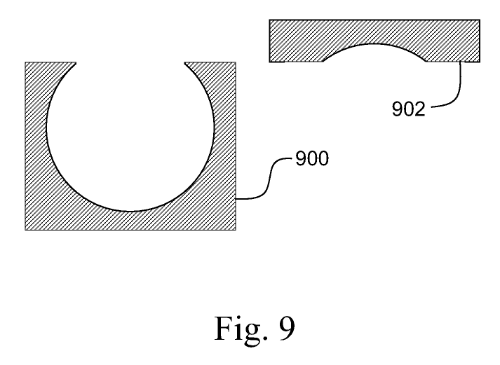

FIGS. 8A and 8B show an embodiment of a system 802 that includes drop generator system 601, and that can be used to make particles. System 802 includes drop generator system 701, a reactor vessel 830, a gel dissolution chamber 840 and a filter 850. During use of system 802, flow controller 700 delivers a solution that contains the polymer matrix and a gelling precursor (e.g., alginate) to viscosity controller 705, which heats the solution to reduce viscosity prior to delivery to drop generator 710. The solution passes through an orifice in a nozzle in drop generator 710, forming drops of the solution. The drops are then directed into vessel 720 (in this process, used as a gelling vessel), where the drops contact a gelling agent (e.g., calcium chloride) that converts the gelling precursor from a solution form into a gel form, stabilizing the drops and forming particles. In some embodiments, the particles can be transferred from vessel 720 to reactor vessel 830, where the polymer matrix in the gel-stabilized particles can be reacted (e.g., cross-linked). In certain embodiments in which the gel-stabilized particles include a polymer including vinyl formal monomer units, the polymer can be further formalized in reactor vessel 830 (so that the weight percent of vinyl formal monomer units in the polymer increases). In some embodiments (e.g., embodiments in which the polymer in the solution already has a desired amount of cross-linking), the particles may not be transferred to reactor vessel 830. In certain embodiments, the particles can be transferred to gel dissolution chamber 840, where the gelling precursor (which was converted to a gel) can be removed from the particles. In some embodiments, the removal of the gel from the particles can result in the formation of pores in the particles. After they have been formed, the particles can be filtered in filter 850 to remove debris. In certain embodiments, the particles can thereafter be coated with, for example, a polymer (e.g., a polyvinyl alcohol). In some embodiments, the coating can be added to the particles by spraying and/or dip-coating. These coating processes can be used, for example, to form particles such as particles 400 or 500 (see FIGS. 4 and 5). Finally, the particles can be sterilized and packaged as, for example, an embolic composition including the particles.

While alginate has been described as a gelling precursor, other types of gelling precursors can be used. Gelling precursors include, for example, alginate salts, xanthan gums, natural gum, agar, agarose, chitosan, carrageenan, fucoidan, furcellaran, laminaran, hypnea, eucheuma, gum arabic, gum ghatti, gum karaya, gum tragacanth, hyaluronic acid, locust beam gum, arabinogalactan, pectin, amylopectin, other water soluble polysaccharides and other ionically cross-linkable polymers. A particular gelling precursor is sodium alginate, such as high guluronic acid, stem-derived alginate (e.g., 50 percent or more, 60 percent or more guluronic acid) with a low viscosity (e.g., from 20 centipoise to 80 centipoise at 20° C.), which can produce a high tensile, robust gel.

As described above, in some embodiments (e.g., some embodiments in which alginate is used as a gelling precursor), vessel 720 can include a gelling agent such as calcium chloride. The calcium cations in the calcium chloride have an affinity for carboxylic groups in the gelling precursor. In some embodiments, the cations can complex with carboxylic groups in the gelling precursor. Without wishing to be bound by theory, it is believed that the complexing of the cations with carboxylic groups in the gelling precursor can cause different regions of the gelling precursor to be pulled closer together, causing the gelling precursor to gel. In certain embodiments, the complexing of the cations with carboxylic groups in the gelling precursor can result in encapsulation of one or more other polymers (e.g., a polymer including vinyl formal monomer units) in a matrix of gelling precursor.

While calcium chloride has been described as a gelling agent, other types of gelling agents can be used. Examples of gelling agents include divalent cations such as alkali metal salts, alkaline earth metal salts, or transition metal salts that can ionically cross-link with the gelling precursor. In some embodiments, an inorganic salt, such as a calcium, barium, zinc or magnesium salt, can be used as a gelling agent.

As discussed above, in certain embodiments, the particles can be transferred to reactor vessel 830 during the particle formation process, where the polymers in the polymer matrix of the particles can, for example, be cross-linked by one or more cross-linking agents. Examples of cross-linking agents that may be used in reactor vessel 830 include one or more aldehydes (e.g., formaldehyde, glyoxal, benzaldehyde, aterephthalaldehyde, succinaldehyde, glutaraldehyde) in combination with one or more acids, such as relatively strong acids (e.g., sulfuric acid, hydrochloric acid, nitric acid) and/or relatively weak acids (e.g., acetic acid, formic acid, phosphoric acid).

In some embodiments, it can be desirable to reduce the surface tension of the mixture contained in vessel 720 (e.g., when forming particles having a maximum dimension of 500 microns or less). This can be achieved, for example, by heating the mixture in vessel 720 (e.g., to a temperature greater than room temperature, such as a temperature of 30° C. or more), by bubbling a gas (e.g., air, nitrogen, argon, krypton, helium, neon) through the mixture contained in vessel 720, by stirring (e.g., via a magnetic stirrer) the mixture contained in vessel 720, by including a surfactant in the mixture containing the gelling agent, and/or by forming a mist containing the gelling agent above the mixture contained in vessel 720 (e.g., to reduce the formation of tails and/or enhance the sphericity of the particles).

In certain embodiments, particles can be formed by omitting one or more of the steps from the process described with reference to FIGS. 8A and 8B. For example, one or more of the polymers may not be crosslinked, and/or the gelling precursor may not be removed.

In some embodiments, particles can be formed by a solvent evaporation method. First, a polymer solution can be formed by dissolving a polymer matrix in a solvent (e.g., dichloromethane, chloroform, benzene, toluene). Then, the polymer solution can be poured as a stream into an aqueous solution including a surfactant (e.g., polyvinyl alcohol) under stirring. The interfacial tension between the polymer solution and the aqueous solution can result in the formation of particles having relatively good sphericity. The solvent can be evaporated over time, thereby causing the particles to harden. The resulting particles can be relatively hard while also being relatively compressible, and/or can have relatively little porosity.

Methods of forming particles are described in, for example, Song et al., U.S. patent application Ser. No. 11/314,056, filed on Dec. 21, 2005 and entitled “Block Copolymer Particles”; Song et al., U.S. patent application Ser. No. 11/314,557, filed on Dec. 21, 2005 and entitled “Block Copolymer Particles”; Buiser et al., U.S. Patent Application Publication No. US 2003/0185896 A1, published on Oct. 2, 2003, and entitled “Embolization”; Lanphere et al., U.S. Patent Application Publication No. US 2004/0096662 A1, published on May 20, 2004, and entitled “Embolization”; Lanphere et al., U.S. Patent Application Publication No. US 2005/0263916 A1, published on Dec. 1, 2005, and entitled “Embolization”; and DiCarlo et al., U.S. patent application Ser. No. 11/111,511, filed on Apr. 21, 2005, and entitled “Particles”, all of which are incorporated herein by reference.

In some embodiments, one or more of the therapeutic agents can be omitted from the processes described above with respect to FIGS. 6A-6C, 7, and/or 8A-8B. In certain embodiments, all of the therapeutic agents can be omitted from a particle formation process, such that the particles that are produced do not include any therapeutic agent. Alternatively or additionally, one or more therapeutic agents can be added to the particles (e.g., by adsorption, by absorption) after the particles have been formed. The therapeutic agents can be incorporated into the particles by, for example, immersing the particles in the therapeutic agents (e.g., in a solution including the therapeutic agents), and/or spraying the particles with the therapeutic agents (e.g., with a solution including the therapeutic agents). Immersing the particles in the therapeutic agents can allow the particles to imbibe the therapeutic agents. In some embodiments in which the particles are porous, the porosity of the particles can, for example, help the therapeutic agents to diffuse into the particles and/or help the particles to retain a relatively high volume of the therapeutic agents. In certain embodiments, the therapeutic agents can be incorporated into the particles by coating the particles with the therapeutic agents. In some embodiments, the therapeutic agents can be incorporated into the particles by covalently bonding the therapeutic agents to one or more of the materials (e.g., polymers) out of which the particles are formed. For example, in some embodiments in which a particle includes a polymer including vinyl formal monomer units and having pendant hydroxyl groups, the pendant hydroxyl groups can be used to form ester linkages with one or more therapeutic agents (e.g., acidic therapeutic agents). In certain embodiments, the therapeutic agents can be incorporated into the particles by spray-drying and/or coating the particles with the therapeutic agents in a fluidized bed dryer. In some embodiments, particles that are coated with a therapeutic agent in a fluidized bed dryer can have a relatively even coating of the therapeutic agent and/or can be relatively unlikely to stick to each other during the coating process.

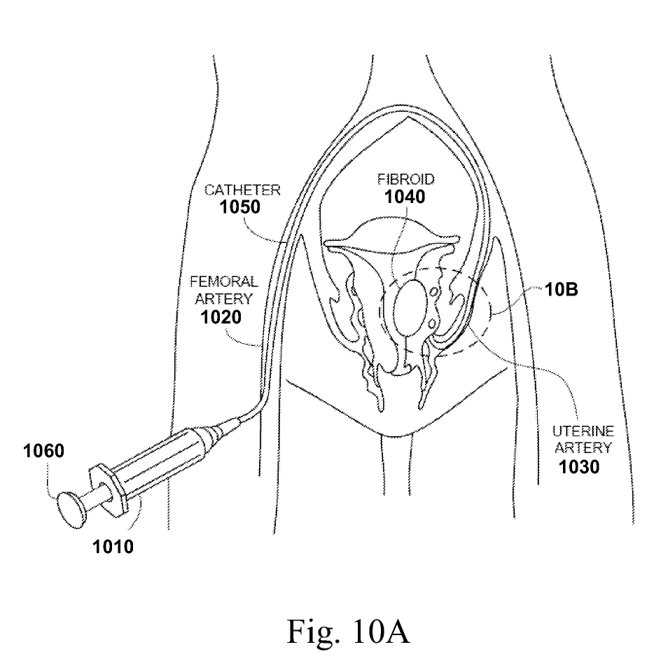

A further method for making a particle involves forming the particle in a mold. FIG. 9 shows a mold 900 that can be used to make a particle. In this method, a polymer matrix as described above is placed in the mold in a condition that will allow the matrix to conform to the shape of the interior surface of the mold. Once enough polymer matrix is placed in the mold 900, a mold lid 902 optionally can be placed on the mold 900 to complete the particle shape. The polymer matrix can be placed in a condition that will allow it to conform to the shape of the interior of the mold in a variety of ways that are well known to those of skill in the art. Such processes include, but are not limited to, extrusion and heating the mold 902 to melt non-molten polymer matrix placed in the mold 900.

In general, a mold 900 can have a largest dimension of 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the largest dimension of mold 900 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the largest dimension of the mold 900 is less than 100 microns (e.g., less than 50 microns).

A method for making a composition for the controlled release of a therapeutic agent involves combining a first polymer and a second polymer to form a composition. The first polymer is one of polyvinyl alcohol or polyvinyl formal. The second polymer is one of polyvinyl alcohol, polyvinyl formal, polyvinylpyrrolidone, polysaccharide, or polymethacrylate. The first polymer and the second polymer of the composition are different and different ratios of the first polymer and the second polymer provide different rates of release of a therapeutic agent from the composition. A third polymer can optionally be combined with the first polymer and second polymer. The third polymer can be one of polyvinyl alcohol, polyvinyl formal, polyvinylpyrrolidone, polysaccharide, or polymethacrylate. In a three polymer composition, the first polymer, second polymer, and third polymer are different. The therapeutic agent can be added at various stages during the method, such as before the polymers are combined, at the time the polymers are combined, after the composition is formed.

Particles can be made from this composition using, but not limited to, the techniques outlined above. Other types of medical devices or drug delivery devices can also be formed from these compositions. The compositions can also be used to coat medical devices. Methods for making medical devices or drug delivery devices are well known to those of skill in the art and can include, for example, techniques such as extrusion and injection molding. Methods for coating a medical device or drug delivery device also are well known to those of skill in the art and can include, for example, techniques such as dipping and spray coating. As discussed above for particles, therapeutic agents can also be included in the polymer matrix before, during, or after formation into a non-particle device or coating.

Further embodiments of these methods can include the additional step of combining the particles, coatings, or devices with pharmaceutically acceptable media, therapeutic agents, radiopaque materials, materials that are visible by magnetic resonance imaging (MRI-visible materials), ferromagnetic materials, and/or contrast agents (e.g., ultrasound contrast agents).

Additional embodiments of these methods can include connecting a particle that is formed to a second particle by forming a link between the particles. Particle chains and methods of making particle chains are described, for example, in Buiser et al., U.S. Patent Application Publication No. US 2005/0238870 A1, published on Oct. 27, 2005, and entitled “Embolization,” which is incorporated herein by reference.

The same polymers, additives (such as waxes and alginate), and therapeutic agents useful with the particle 100 described above are useful with these methods. The physical characteristics, i.e., largest dimension, length, aspect ratio, etc., of particles and particle chains formed from particles made by these methods are the same as those described above for particle chain 110.

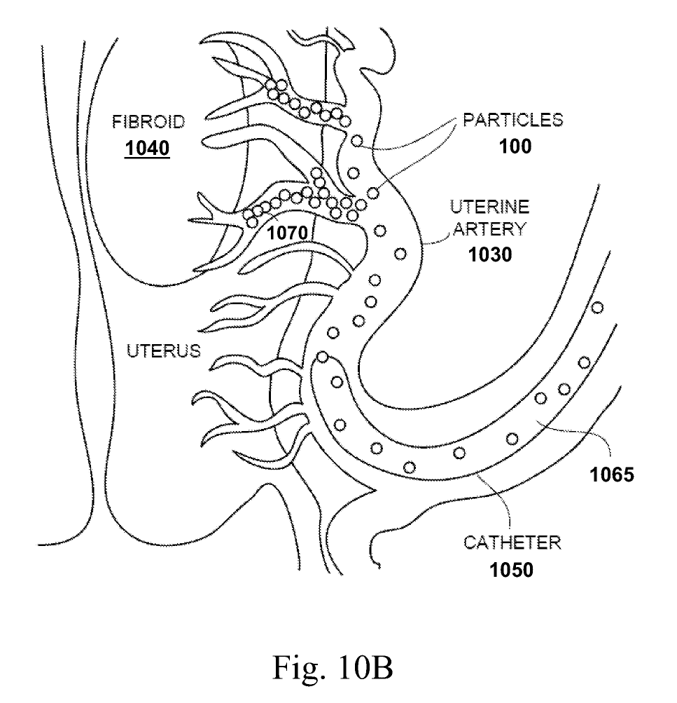

In some embodiments, in addition to or as an alternative to being used to deliver a therapeutic agent to a target site, the particle 100 or particle chain 110 can be used to embolize a target site (e.g., a lumen of a subject). For example, multiple particles can be combined with a carrier fluid (e.g., a pharmaceutically acceptable carrier, such as a saline solution, a contrast agent, or both) to form a composition, which can then be delivered to a site and used to embolize the site. FIGS. 10A and 10B illustrate the use of a composition including particles to embolize a lumen of a subject. As shown, a composition, including particles 100 or particle chain 110 and a carrier fluid, is injected into a vessel through an instrument such as a catheter 1050. Catheter 1050 is connected to a syringe barrel 1010 with a plunger 1060. Catheter 1050 is inserted, for example, into a femoral artery 1020 of a subject. Catheter 1050 delivers the composition to, for example, occlude a uterine artery 1030 leading to a fibroid 1040. Fibroid 1040 is located in the uterus of a female subject. The composition is initially loaded into syringe 1010. Plunger 1060 of syringe 1010 is then compressed to deliver the composition through catheter 1050 into a lumen 1065 of uterine artery 1030.

FIG. 10B, which is an enlarged view of section 10B of FIG. 10A, shows a uterine artery 1030 that is subdivided into smaller uterine vessels 1070 (e.g., having a largest dimension of two millimeters or less) which feed fibroid 1040. The particles 100 or particle chain 110 in the composition partially or totally fill the lumen of uterine artery 1030, either partially or completely occluding the lumen of the uterine artery 1130 that feeds uterine fibroid 1140.

Compositions that include particles such as particles 100 or particle chain 110 can be delivered to various sites in the body, including, for example, sites having cancerous lesions, such as the breast, prostate, lung, thyroid, or ovaries. The compositions can be used in, for example, neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are for example, abnormal collections of blood vessels, e.g. in the brain, which shunt blood from a high pressure artery to a low pressure vein, resulting in hypoxia and malnutrition of those regions from which the blood is diverted. In some embodiments, a composition containing the particles can be used to prophylactically treat a condition.

The magnitude of a dose of a composition can vary based on the nature, location and severity of the condition to be treated, as well as the route of administration. A physician treating the condition, disease or disorder can determine an effective amount of composition. An effective amount of embolic composition refers to the amount sufficient to result in amelioration of symptoms and/or a prolongation of survival of the subject, or the amount sufficient to prophylactically treat a subject. The compositions can be administered as pharmaceutically acceptable compositions to a subject in any therapeutically acceptable dosage, including those administered to a subject intravenously, subcutaneously, percutaneously, intratrachealy, intramuscularly, intramucosaly, intracutaneously, intra-articularly, orally or parenterally.

A composition can include a mixture of particles or particle chains (e.g., particles or particle chains that include different types of polymers, particles that include different types of therapeutic agents), or can include particles that are all of the same type. In some embodiments, a composition can be prepared with a calibrated concentration of particles or particle chains for ease of delivery by a physician. A physician can select a composition of a particular concentration based on, for example, the type of procedure to be performed. In certain embodiments, a physician can use a composition with a relatively high concentration of particles or particle chains during one part of an embolization procedure, and a composition with a relatively low concentration of particles or particle chains during another part of the embolization procedure.

Suspensions of particles or particle chains in saline solution can be prepared to remain stable (e.g., to remain suspended in solution and not settle and/or float) over a desired period of time. A suspension of particles or particle chains can be stable, for example, for from one minute to 20 minutes (e.g. from one minute to 10 minutes, from two minutes to seven minutes, from three minutes to six minutes).

In some embodiments, particles or particle chains can be suspended in a physiological solution by matching the density of the solution to the density of the particles or particle chains. In certain embodiments, the particles or particle chains and/or the physiological solution can have a density of from one gram per cubic centimeter to 1.5 grams per cubic centimeter (e.g., from 1.2 grams per cubic centimeter to 1.4 grams per cubic centimeter, from 1.2 grams per cubic centimeter to 1.3 grams per cubic centimeter).

In some embodiments, the carrier fluid of a composition can include a surfactant. The surfactant can help the particles or particle chains to mix evenly in the carrier fluid and/or can decrease the likelihood of the occlusion of a delivery device (e.g., a catheter) by the particles. In certain embodiments, the surfactant can enhance delivery of the composition (e.g., by enhancing the wetting properties of the particles or particle chains and facilitating the passage of the particles through a delivery device). In some embodiments, the surfactant can decrease the occurrence of air entrapment by the particles or particle chains in a composition (e.g., by porous particles in a composition). Examples of liquid surfactants include Tween® 80 (available from Sigma-Aldrich) and Cremophor EL® (available from Sigma-Aldrich). An example of a powder surfactant is Pluronic® F127 NF (available from BASF). In certain embodiments, a composition can include from 0.05 percent by weight to one percent by weight (e.g., 0.1 percent by weight, 0.5 percent by weight) of a surfactant. A surfactant can be added to the carrier fluid prior to mixing with the particles or particle chains and/or can be added to the particles or particle chains prior to mixing with the carrier fluid.

In some embodiments, among the particles delivered to a subject (e.g., in a composition), the majority (e.g., 50 percent or more, 60 percent or more, 70 percent or more, 80 percent or more, 90 percent or more) of the particles can have a largest dimension of 5,000 microns or less (e.g., 4,500 microns or less; 4,000 microns or less; 3,500 microns or less; 3,000 microns or less; 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, among the particles delivered to a subject, the majority of the particles can have a largest dimension of less than 100 microns (e.g., less than 50 microns).

In certain embodiments, the particles delivered to a subject (e.g., in a composition) can have an arithmetic mean largest dimension of 5,000 microns or less (e.g., 4,500 microns or less; 4,000 microns or less; 3,500 microns or less; 3,000 microns or less; 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the particles delivered to a subject can have an arithmetic mean largest dimension of less than 100 microns (e.g., less than 50 microns).

Exemplary ranges for the arithmetic mean largest dimension of particles or particle portions of particle chains delivered to a subject include from 100 microns to 500 microns; from 100 microns to 300 microns; from 300 microns to 500 microns; from 500 microns to 700 microns; from 700 microns to 900 microns; from 900 microns to 1,200 microns; and from 1,000 microns to 1,200 microns. In general, the particles or particle portions of particle chains delivered to a subject (e.g., in a composition) can have an arithmetic mean largest dimension in approximately the middle of the range of the largest dimensions of the individual particles or particle portions of particle chains, and a variance of 20 percent or less (e.g. 15 percent or less, 10 percent or less).

In some embodiments, the arithmetic mean largest dimension of the particles or particle portions of particle chains delivered to a subject (e.g., in a composition) can vary depending upon the particular condition to be treated. As an example, in embodiments in which the particles or particle chains are used to embolize a liver tumor, the particles or particle portions of particle chains delivered to the subject can have an arithmetic mean largest dimension of 500 microns or less (e.g., from 100 microns to 300 microns; from 300 microns to 500 microns). As another example, in embodiments in which the particles or particle chains are used to embolize a uterine fibroid, the particles or particle portions of particle chains delivered to the subject can have an arithmetic mean largest dimension of 1,200 microns or less (e.g., from 500 microns to 700 microns; from 700 microns to 900 microns; from 900 microns to 1,200 microns). As an additional example, in embodiments in which the particles or particle chains are used to treat a neural condition (e.g., a brain tumor) and/or head trauma (e.g., bleeding in the head), the particles or particle portions of particle chains delivered to the subject can have an arithmetic mean largest dimension of less than 100 microns (e.g., less than 50 microns). As a further example, in embodiments in which the particles or particle chains are used to treat a lung condition, the particles or particle portions of particle chains delivered to the subject can have an arithmetic mean largest dimension of less than 100 microns (e.g., less than 50 microns). As another example, in embodiments in which the particles or particle chains are used to treat thyroid cancer, the particles or particle portions of particle chains can have a largest dimension of 1,200 microns or less (e.g., from 1,000 microns to 1,200 microns). As an additional example, in some embodiments in which the particles are used only for therapeutic agent delivery, the particles can have an arithmetic mean maximum dimension of less than 100 microns (e.g., less than 50 microns, less than 10 microns, less than five microns).

The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the diameters of all of the particles in the group by the number of particles in the group.

### EXAMPLES

### Example 1

### Polyvinyl Alcohol and Polyvinyl Formal Therapeutic Agent Release Rates

To determine the base therapeutic agent release rates for polyvinyl alcohol (99% hydrolyzed) (Sigma-Aldrich, Co.; St. Louis, Mo.) and polyvinyl formal (SPI Supplies® (West Chester, Pa.)), polymer films containing ketorolac tromethiamine (Spectrum Chemical Mfg. Corp.; Gardena, Calif.) were created.

4 g of polyvinyl alcohol was added to 50 mL of deionized water, then the mixture was heated and homogenized to create an 8% w/v polyvinyl alcohol solution. 4 g of polyvinyl formal was added to 50 mL of glacial acetic acid with continuous stirring to create an 8% w/v polyvinyl formal solution. Ketorolac tromathiamine was added to each polymer solution such that the concentration was 5% w/v. 0.5 mL of each solution was spread evenly over 3 numbered glass slides (6 slides total) and dried for one hour in an incubator at 37° C.

The slides were each placed in tubes containing 40 mL of PBS-Tween 20 media (Sigma; St. Louis, Mo.). 0.5 mL samples were taken from each slide's tube at the following time intervals: 0, 2, 4, 6, 24, and 48 hours. After each sample was removed, 0.5 mL of fresh media was added to the tubes. Between samples, the tubes were kept at 37° C. in an incubator-shaker (1500 rpm). Samples were measured by HPLC (Waters Corp., Milford, Mass.) using a Waters 2695 Separations Module, a Waters 2487 Dual Absorbance Detector, and a Waters 2996 PDA Detector with the Waters Empower software. HPLC conditions and parameters were as follows:

| | |
|---|---|
| Gradient Profile | Isocratic |
| Analytical Column: | XTERRA MS C18 or equivalent |
| Pre column filter | MACMOD, |
| Guard column | Nova-Pak C18, or equivalent |
| Column Temp | 35° C. |
| Mobile Phase | 70:30 (2% Acetic Acid:Acetonitrile) |
| Flow Rate | 1 ml/min |
| Injection Volume | 50 μL |
| Detection Wavelength | 313 nm |
| Sampling Rate | 5 points/second |

The average cumulative and average percentage ketorolac releases are for polyvinyl alcohol are provided below in Table 1 (for 3 samples). The data in Table 1 is shown in graphical form in FIG. 2.

TABLE 1
Total Ketorolac Tromethamine Release From PVA Over Time

| | |
|---|---|
| Ketorolac | |
| Release | % Ketorolac |
| (mg/mL) | Release |

| Time | Avg. | St. Dev. | Avg. | St. Dev. |
|---|---|---|---|---|
| 0 | 5.61 | 1.87 | 22.44% | 7.49% |
| 2 | 23.78 | 3.26 | 95.10% | 13.02% |
| 4 | 23.89 | 3.38 | 95.56% | 13.50% |
| 6 | 24.50 | 3.15 | 97.99% | 12.62% |
| 24 | 23.76 | 3.38 | 95.03% | 13.52% |
| 48 | 25.60 | 1.37 | 102.40% | 5.49% |

The average cumulative and average percentage ketorolac releases are for polyvinyl formal are provided below in Table 2 (for 3 samples). The data in Table 2 is shown in graphical form in FIG. 3.

TABLE 2
Total Ketorolac Tromethamine Release From PVF Over Time

| | |
|---|---|
| Ketorolac | % Ketorolac |
| Release (mg) | Release |

| Time | Avg. | St. Dev. | Avg. | St. Dev. |
|---|---|---|---|---|
| 0 | 0.38 | 0.65 | 3.01% | 5.21% |
| 2 | 1.76 | 0.21 | 7.03% | 0.82% |
| 4 | 2.01 | 0.24 | 8.05% | 0.97% |
| 6 | 2.21 | 0.22 | 8.86% | 0.87% |
| 24 | 4.43 | 0.50 | 17.73% | 2.02% |
| 48 | 6.45 | 0.63 | 25.80% | 2.53% |

As can be readily seen from the data, polyvinyl alcohol releases ketorolac tromethamine quickly with over 95% released in the first two hours. Whereas polyvinyl formal releases ketorolac tromethamine much more slowly with only about 25% release at 48 hours.

### Example 2

### Therapeutic Agent Release Rates for Multiple Polymer Compositions

Sample slides were prepared as in Example 1 with polyvinyl alcohol/ketorolac tromethamine or polyvinyl formal/ketorolac tromethane layers and dried. Then 0.5 mL of an additional polymer solution (with the parameters shown in Table 3) was applied to the slides and the slides were dried for one hour in an incubator at 37° C. The additional polymer solutions were prepared in 50 mL of deionized water to provide the w/v solutions referred to in Table 3. The release protocol and analysis by HPLC was the same as for Example 1.

TABLE 3
Example 3 Polymer Compositions

| Base Layer | 2^{nd} Polymer | # of Samples |
|---|---|---|
| PVA/Ketorolac | 0.1% PVP^{a} w/v | 3 |
| PVA/Ketorolac | 0.2% PVP w/v | 3 |
| PVA/Ketorolac | Eudragit NE 30D^{b} | 2 |
| PVA/Ketorolac | Eudragit RS 30D^{c} | 3 |
| PVF/Ketorolac | 0.1% PVP w/v | 3 |
| PVF/Ketorolac | 0.2% PVP w/v | 3 |
| PVF/Ketorolac | Eudragit NE 30D | 3 |
| PVF/Ketorolac | Eudragit RS 30D | 1 |

^{a}PVP (polyvinylpyrrolidone): Kollidone 90F from Spectrum Chemical Mfg. Corp. (Gardena, CA).
^{b}Eudragit NE 30D 30% dispersion from Degussa Corp. (Parsippany, NJ).
^{c}Eudragit RS 30D 30% dispersion from Degussa Corp. (Parsippany, NJ).

The release data for the polyvinyl alcohol compositions listed in Table 3 is provided in Table 4 through Table 7 below:

TABLE 4
Total Ketorolac Tromethamine Release From PVA + 0.1% PVP Over
Time

| | |
|---|---|
| Ketorolac | % Ketorolac |
| Release (mg) | Release |

| Time | Avg. | St. Dev. | Avg. | St. Dev. |
|---|---|---|---|---|
| 0 | 1.80 | 1.08 | 7.21% | 4.33% |
| 2 | 22.75 | 1.52 | 91.02% | 6.08% |
| 4 | 22.50 | 1.53 | 89.98% | 6.11% |
| 6 | 22.50 | 1.61 | 90.02% | 6.46% |
| 24 | 23.04 | 1.84 | 92.17% | 7.38% |
| 48 | 23.23 | 2.26 | 92.92% | 9.03% |

TABLE 5
Total Ketorolac Tromethamine Release From PVA + 0.2% PVP Over
Time

| | |
|---|---|
| Ketorolac | % Ketorolac |
| Release (mg) | Release |

| Time | Avg. | St. Dev. | Avg. | St. Dev. |
|---|---|---|---|---|
| 0 | 1.02 | 0.72 | 4.08% | 2.86% |
| 2 | 19.76 | 2.84 | 79.05% | 11.37% |
| 4 | 19.74 | 3.39 | 78.96% | 13.57% |
| 6 | 19.22 | 2.81 | 76.86% | 11.23% |
| 24 | 19.80 | 3.01 | 79.19% | 12.04% |
| 48 | 19.92 | 3.72 | 79.69% | 14.89% |

TABLE 6
Total Ketorolac Tromethamine Release From PVA +
Eudragit NE 30D Over Time

| | |
|---|---|
| Ketorolac | % Ketorolac |
| Release (mg) | Release |

| Time | Avg. | St. Dev. | Avg. | St. Dev. |
|---|---|---|---|---|
| 0 | 0.78 | 0.79 | 3.13% | 3.17% |
| 2 | 25.19 | 1.80 | 100.75% | 7.19% |
| 4 | 24.84 | 1.97 | 99.35% | 7.88% |
| 6 | 25.27 | 1.88 | 101.10% | 7.50% |
| 24 | 25.16 | 2.04 | 100.63% | 8.15% |
| 48 | 25.20 | 1.74 | 100.81% | 6.95% |

TABLE 7
Total Ketorolac Tromethamine Release From PVA + Eudragit
RS 30D Over Time

| | |
|---|---|
| Ketorolac | % Ketorolac |
| Release (mg) | Release |

| Time | Avg. | St. Dev. | Avg. | St. Dev. |
|---|---|---|---|---|
| 0 | 0.86 | 0.55 | 3.44% | 2.20% |
| 2 | 21.69 | 7.50 | 86.75% | 29.98% |
| 4 | 24.32 | 3.53 | 97.29% | 14.10% |
| 6 | 24.34 | 2.93 | 97.34% | 11.72% |
| 24 | 24.23 | 3.25 | 96.90% | 12.99% |
| 48 | 24.64 | 2.95 | 98.56% | 11.80% |

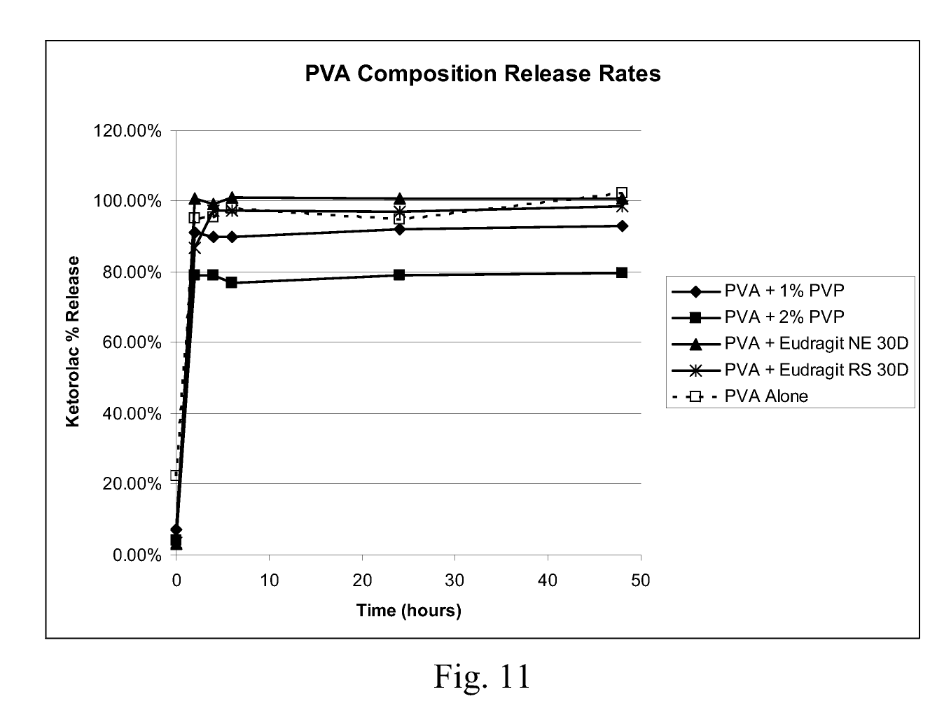

FIG. 11 shows a plot of the polyvinyl alcohol data listed in Table 4 through Table 7. For comparison purposes, the ketorolac release rate for polyvinyl alcohol alone is included in FIG. 11. As can be seen from the data, the burst release rate of ketorolac release from a polyvinyl alcohol base polymer can be decreased to less than 80% followed by gradual release for an extended period of time by adding 2% PVP and by various other amounts by adding different polymers.

The release data for the polyvinyl formal compositions listed in Table 3 is provided in Table 8 through Table 11 below:

TABLE 8
Total Ketorolac Tromethamine Release From PVF + 0.1% PVP Over
Time

| | |
|---|---|
| Ketorolac | % Ketorolac |
| Release (mg) | Release |

| Time | Avg. | St. Dev. | Avg. | St. Dev. |
|---|---|---|---|---|
| 0 | 0.38 | 0.66 | 3.04% | 5.27% |
| 2 | 1.77 | 0.21 | 7.09% | 0.82% |
| 4 | 2.07 | 0.21 | 8.27% | 0.86% |
| 6 | 2.29 | 0.29 | 9.16% | 1.16% |
| 24 | 4.88 | 0.53 | 19.53% | 2.11% |
| 48 | 8.37 | 0.64 | 33.49% | 2.54% |

TABLE 9
Total Ketorolac Tromethamine Release From PVF + 0.2% PVP Over
Time

| | |
|---|---|
| Ketorolac | % Ketorolac |
| Release (mg) | Release |

| Time | Avg. | St. Dev. | Avg. | St. Dev. |
|---|---|---|---|---|
| 0 | 0.07 | 0.13 | 0.60% | 1.04% |
| 2 | 1.81 | 0.06 | 7.24% | 0.25% |
| 4 | 2.13 | 0.03 | 8.50% | 0.12% |
| 6 | 2.39 | 0.08 | 9.55% | 0.31% |
| 24 | 6.21 | 1.69 | 24.85% | 6.77% |
| 48 | 9.63 | 3.29 | 38.51% | 13.16% |

TABLE 10
Total Ketorolac Tromethamine Release From PVF + Eudragit
NE 30D Over Time

| | |
|---|---|
| Ketorolac | % Ketorolac |
| Release (mg) | Release |

| Time | Avg. | St. Dev. | Avg. | St. Dev. |
|---|---|---|---|---|
| 0 | 0.77 | 0.67 | 6.13% | 5.32% |
| 2 | 1.68 | 0.20 | 6.72% | 0.79% |
| 4 | 2.20 | 0.59 | 8.79% | 2.36% |
| 6 | 2.53 | 0.76 | 10.13% | 3.05% |
| 24 | 7.84 | 1.84 | 31.37% | 7.36% |
| 48 | 12.17 | 1.46 | 48.69% | 5.85% |

TABLE 11
Total Ketorolac Tromethamine Release From PVF + Eudragit
RS 30D Over Time

| | |
|---|---|
| Ketorolac | % Ketorolac |
| Release (mg) | Release |

| Time | Value | St. Dev. | Value | St. Dev. |
|---|---|---|---|---|
| 0 | 0.00 | — | 0.00% | — |
| 2 | 3.60 | — | 14.40% | — |
| 4 | 4.70 | — | 18.81% | — |
| 6 | 5.33 | — | 21.33% | — |
| 24 | 13.09 | — | 52.37% | — |
| 48 | 15.43 | — | 61.74% | — |

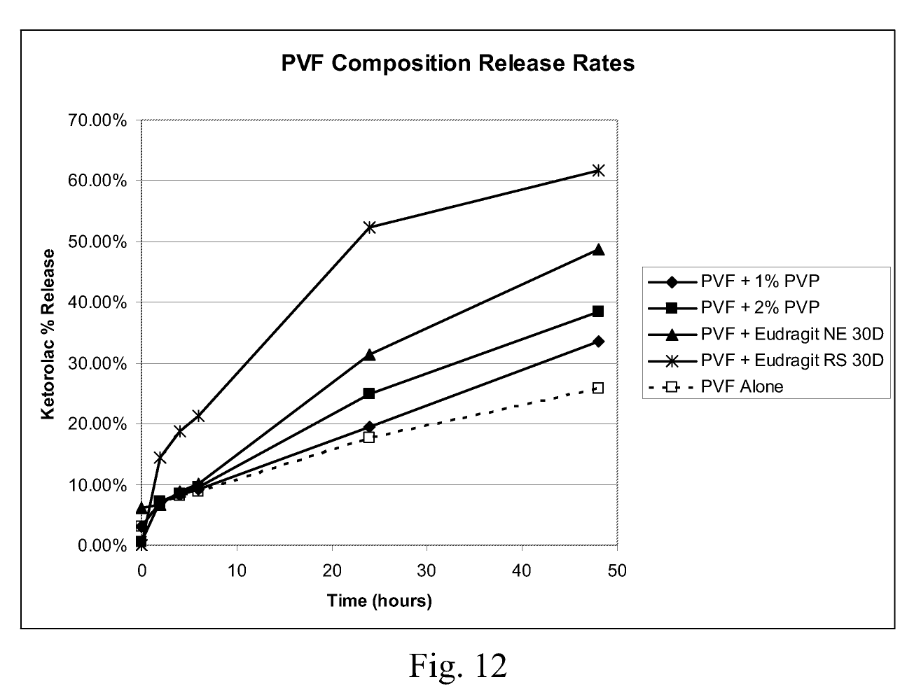

FIG. 12 shows a plot of the polyvinyl formal data listed in Table 8 through Table 11. For comparison purposes, the ketorolac release rate for polyvinyl formal alone is included in FIG. 12. As can be seen from the data, the rate of ketorolac release from a polyvinyl formal base polymer can be increased to more than 60% in 48 hours by adding Eudragit RS 30D as compared to approximately 25% with polyvinyl formal alone, and by various other amounts by adding different polymers.

Overall these data demonstrate that polyvinyl alcohol and polyvinyl formal compositions including additional polymers have different rates of release for therapeutic agents.

### Other Embodiments

While certain embodiments have been described, other embodiments are possible.

As an example, in some embodiments in which a particle is used for embolization, the particle can also include one or more other embolic agents, such as a sclerosing agent (e.g., ethanol), a liquid embolic agent (e.g., n-butyl-cyanoacrylate), and/or a fibrin agent. The other embolic agent(s) can enhance the restriction of blood flow at a target site.

As another example, in some embodiments, a particle as described herein can also include a surface preferential material. Surface preferential materials are described, for example, in DiCarlo et al., U.S. Patent Application Publication No. US 2005/0196449 A1, published on Sep. 8, 2005, and entitled “Embolization”, which is incorporated herein by reference.

As an additional example, in some embodiments one or more particles is/are substantially nonspherical. In some embodiments, particles can be mechanically shaped during or after the particle formation process to be nonspherical (e.g., ellipsoidal). In certain embodiments, particles can be shaped (e.g., molded, compressed, punched, and/or agglomerated with other particles) at different points in the particle manufacturing process. As an example, in some embodiments, the particles can be sufficiently flexible and/or moldable to be shaped. As another example, in certain embodiments in which particles are formed using a gelling agent, the particles can be physically deformed into a specific shape and/or size after the particles have been contacted with the gelling agent, but before the polymer(s) in the particles have been cross-linked. After shaping, the polymer(s) (e.g., polyvinyl alcohol) in the particles can be cross-linked, optionally followed by substantial removal of gelling precursor (e.g., alginate). While substantially spherical particles have been described, in some embodiments, nonspherical particles can be manufactured and formed by controlling, for example, drop formation conditions. In some embodiments, nonspherical particles can be formed by post-processing the particles (e.g., by cutting or dicing into other shapes). Particle shaping is described, for example, in Baldwin et al., U.S. Patent Application Publication No. US 2003/0203985 A1, published on Oct. 30, 2003, and entitled “Forming a Chemically Cross-Linked Particle of a Desired Shape and Diameter”, which is incorporated herein by reference.

As a further example, in some embodiments, particles can be used for tissue bulking. As an example, the particles can be placed (e.g., injected) into tissue adjacent to a body passageway. The particles can narrow the passageway, thereby providing bulk and allowing the tissue to constrict the passageway more easily. The particles can be placed in the tissue according to a number of different methods, for example, percutaneously, laparoscopically, and/or through a catheter. In certain embodiments, a cavity can be formed in the tissue, and the particles can be placed in the cavity. Particle tissue bulking can be used to treat, for example, intrinsic sphincteric deficiency (ISD), vesicoureteral reflux, gastroesophageal reflux disease (GERD), and/or vocal cord paralysis (e.g., to restore glottic competence in cases of paralytic dysphonia). In some embodiments, particle tissue bulking can be used to treat urinary incontinence and/or fecal incontinence. The particles can be used as a graft material or a filler to fill and/or to smooth out soft tissue defects, such as for reconstructive or cosmetic applications (e.g., surgery). Examples of soft tissue defect applications include cleft lips, scars (e.g., depressed scars from chicken pox or acne scars), indentations resulting from liposuction, wrinkles (e.g., glabella frown wrinkles), and soft tissue augmentation of thin lips. Tissue bulking is described, for example, in Bourne et al., U.S. Patent Application Publication No. US 2003/0233150 A1, published on Dec. 18, 2003, and entitled “Tissue Treatment”, which is incorporated herein by reference.

As another example, in some embodiments a solution can be added to the nozzle of a drop generator to enhance the porosity of particles produced by the drop generator. Examples of porosity-enhancing solutions include starch, sodium chloride at a relatively high concentration (e.g., more than 0.9 percent, from one percent to five percent, from one percent to two percent), and calcium chloride (e.g., at a concentration of at least 50 mM). For example, calcium chloride can be added to a sodium alginate gelling precursor solution to increase the porosity of the particles produced from the solution.

As a further example, while certain methods of making particles have been described, in some embodiments, other methods can be used to make particles. For example, in some embodiments (e.g., in some embodiments in which particles having a diameter of one micron or less are being formed), particles can be formed using rotor/stator technology (e.g., Polytron® rotor/stator technology from Kinmatica Inc.), high-pressure homogenization (e.g., using an APV-Gaulin microfluidizer or Gaulin homogenizer), mechanical shear (e.g., using a Gifford Wood colloid mill), and/or ultrasonification (e.g., using either a probe or a flow-through cell).

As an additional example, in some embodiments, particles having different shapes, sizes, physical properties, and/or chemical properties, can be used together in an embolization procedure. The different particles can be delivered into the body of a subject in a predetermined sequence or simultaneously. In certain embodiments, mixtures of different particles can be delivered using a multi-lumen catheter and/or syringe. In some embodiments, particles having different shapes and/or sizes can be capable of interacting synergistically (e.g., by engaging or interlocking) to form a well-packed occlusion, thereby enhancing embolization. Particles with different shapes, sizes, physical properties, and/or chemical properties, and methods of embolization using such particles are described, for example, in Bell et al., U.S. Patent Application Publication No. US 2004/0091543 A1, published on May 13, 2004, and entitled “Embolic Compositions”, and in DiCarlo et al., U.S. Patent Application Publication No. US 2005/0095428 A1, published on May 5, 2005, and entitled “Embolic Compositions”, both of which are incorporated herein by reference.

Other embodiments are in the claims.

1. A particle comprising:
a first polymer selected from the group consisting of polyvinyl alcohol and polyvinyl formal; and
a second polymer selected from the group consisting of polyvinyl alcohol, polyvinyl formal, polyvinylpyrrolidone, a polysaccharide, and a polymethacrylate;
wherein the first polymer and the second polymer are different and the polymers are formed in the shape of a particle.

2. A particle as defined in claim 1, wherein the largest dimension of the particle is at most 5,000 microns.

3. A particle as defined in claim 1, wherein the particle is spherical.

4. A particle as defined in claim 1, further comprising a therapeutic agent.

5. A particle as defined in claim 4, wherein the therapeutic agent is hydrophilic.

6. A particle as defined in claim 4, wherein the therapeutic agent is a chemotherapeutic agent.

7. A particle as defined in claim 4, further comprising a solvent.

8. A particle as defined in claim 7, wherein the solvent is water.

9. A particle as defined in claim 7, wherein the solvent is glacial acetic acid.

10. A particle as defined in claim 1, wherein the percent by weight of the first polymer in the composition is 50 percent or greater.

11. A particle as defined in claim 1, wherein the percent by weight of the first polymer in the composition is 70 percent or greater.

12. A particle as defined in claim 1, wherein the percent by weight of the first polymer in the composition is 90 percent or greater.

13. A particle as defined in claim 1, wherein the percent by weight of the first polymer in the composition is 95 percent or greater.

14. A particle as defined in claim 1, wherein the percent by weight of the first polymer in the composition is 98 percent or greater.

15. A particle as defined in claim 1, wherein the percent by weight of the first polymer in the composition is 99 percent or greater.

16. A particle as defined in claim 1, wherein the percent by weight of the first polymer in the composition is 99.5 percent or greater.

17. A particle as defined in claim 1, wherein the first polymer and second polymer are intimately mixed.

18. A particle as defined in claim 1, wherein the polysaccharide is hydroxylcellulose.

19. A particle as defined in claim 1, wherein the polymethacrylate is a methacrylate copolymer or an ammonioalkyl methacrylate copolymer.

20. A particle as defined in claim 1, further comprising:
a third polymer selected from the group consisting of polyvinyl alcohol, polyvinyl formal, polyvinylpyrrolidone, a polysaccharide, and a polymethacrylate,
wherein the first polymer, second polymer, and third polymer are different.

21. A particle chain comprising a particle as defined in claim 1 connected by a link to at least one other particle.

22. A particle chain as defined in claim 21, wherein the at least one other particle is a particle as defined in claim 1.

23. A particle chain as defined in claim 21, wherein the link is a polymer.

24. A particle chain as defined in claim 21, wherein the link is a metal.

25. A particle chain as defined in claim 21, wherein the link is a fiber.

26-37. (canceled)

38. A composition comprising:
a carrier fluid; and
a plurality of particles within the carrier fluid, the particles comprising
a first polymer selected from the group consisting of polyvinyl alcohol and polyvinyl formal; and
a second polymer selected from the group consisting of polyvinyl alcohol, polyvinyl formal, polyvinylpyrrolidone, a polysaccharide, and a polymethacrylate;
wherein the first polymer and the second polymer are different.

39-55. (canceled)

56. A composition comprising:
a first polymer selected from the group consisting of polyvinyl alcohol and polyvinyl formal;
a second polymer selected from the group consisting of polyvinyl alcohol, polyvinyl formal, polyvinylpyrrolidone, a polysaccharide, and a polymethacrylate; and
a therapeutic agent,
wherein the first polymer and the second polymer are different and different ratios of the first polymer and the second polymer provide different rates of release of the therapeutic agent from the composition.

57-91. (canceled)
